SEQUENCE LISTING (16 sequences)

SEQ ID NO: 1 - protein, 1464 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 2 - protein, 1366 aa, Homo sapiens [sequence omitted]

SEQ ID NO: 3 - protein, 1464 aa, Homo sapiens; identical to SEQ ID NO: 1

SEQ ID NO: 4 - protein, 1366 aa, Homo sapiens [sequence omitted]

SEQ ID NO: 5 - protein, 1489 aa, Homo sapiens
MAHARVLLLALAVLATAAVAVASSSSFADSNPIRPVTDRAASTLAQLQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPAGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKTSRLPIIDVAPLDVGAPDQEFGFDVGPVCFL

SEQ ID NO: 6 - protein, 1389 aa, Homo sapiens
MAHARVLLLALAVLATAAVAVASSSSFADSNPIRPVTDRAASTLAQLLQEETVRKGPAGDRGPRGERGPPGPPGRDGEDGPTGPPGPPGPPGPPGLGGNFAAQYDGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPTGPAGPRGEVGLPGLSGPVGPPGNPGANGLTGAKGAAGLPGVAGAPGLPGPRGIPGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPAGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAHGAVGAPGPAGATGDRGEAGAAGPAGPAGPRGSPGERGEVGPAGPNGFAGPAGAAGQPGAKGERGGKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGFKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRADQPRSAPSLRPKDYEVDATLKSLNNQIETLLTPEGSRKNPARTCRDLRLSHPEWSSGYYWIDPNQGCTMEAIKVYCDFPTGETCIRAQPENIPAKNWYRSSKDKKHVWLGETINAGSQFEYNVEGVTSKEMATQLAFMRLLANYASQNITYHCKNSIAYMDEETGNLKKAVILQGSNDVELVAEGNSRFTYTVLVDGCSKKTNEWGKTIIEYKTNKPSRLPFLDIAPLDIGGADHEFFVDIGPVCFK

SEQ ID NO: 7 - DNA, 5927 bp, Homo sapiens
  source 1..5927: mol_type = genomic DNA
tcgtcggagcagacgggagtttctcctcggggtcggagcaggaggcacgcggagtgtgaggccacgcatgagcggacgctaaccccctccccagccacaaagagtctacatgtctagggtctagacatgttcagctttgtggacctccggctcctgctcctcttagcggccaccgccctcctgacgcacggccaagaggaaggccaagtcgagggccaagacgaagacatcccaccaatcacctgcgtacagaacggcctcaggtaccatgaccgagacgtgtggaaacccgagccctgccggatctgcgtctgcgacaacggcaaggtgttgtgcgatgacgtgatctgtgacgagaccaagaactgccccggcgccgaagtccccgagggcgagtgctgtcccgtctgccccgacggctcagagtcacccaccgaccaagaaaccaccggcgtcgagggacccaagggagacactggcccccgaggcccaaggggacccgcaggcccccctggccgagatggcatccctggacagcctggacttcccggaccccccggaccccccggacctcccggaccccctggcctcggaggaaactttgctccccagctgtcttatggctatgatgagaaatcaaccggaggaatttccgtgcctggccccatgggtccctctggtcctcgtggtctccctggcccccctggtgcacctggtccccaaggcttccaaggtccccctggtgagcctggcgagcctggagcttcaggtcccatgggtccccgaggtcccccaggtccccctggaaagaatggagatgatggggaagctggaaaacctggtcgtcctggtgagcgtgggcctcctgggcctcagggtgctcgaggattgcccggaacagctggcctccctggaatgaagggacacagaggtttcagtggtttggatggtgccaagggagatgctggtcctgctggtcctaagggtgagcctggcagccctggtgaaaatggagctcctggtcagatgggcccccgtggcctgcctggtgagagaggtcgccctggagcccctggccctgctggtgctcgtggaaatgatggtgctactggtgctgccgggccccctggtcccaccggccccgctggtcctcctggcttccctggtgctgttggtgctaagggtgaagctggtccccaagggccccgaggctctgaaggtccccagggtgtgcgtggtgagcctggcccccctggccctgctggtgctgctggccctgctggaaaccctggtgctgatggacagcctggtgctaaaggtgccaatggtgctcctggtattgctggtgctcctggcttccctggtgcccgaggcccctctggaccccagggccccggcggccctcctggtcccaagggtaacagcggtgaacctggtgctcctggcagcaaaggagacactggtgctaagggagagcctggccctgttggtgttcaaggaccccctggccctgctggagaggaaggaaagcgaggagctcgaggtgaacccggacccactggcctgcccggaccccctggcgagcgtggtggacctggtagccgtggtttccctggcgcagatggtgttgctggtcccaagggtcccgctggtgaacgtggttctcctggccctgctggccccaaaggatctcctggtgaagctggtcgtcccggtgaagctggtctgcctggtgccaagggtctgactggaagccctggcagccctggtcctgatggcaaaactggcccccctggtcccgccggtcaagatggtcgccccggacccccaggcccacctggtgcccgtggtcaggctggtgtgatgggattccctggacctaaaggtgctgctggagagcccggcaaggctggagagcgaggtgttcccggaccccctggcgctgtcggtcctgctggcaaagatggagaggctggagctcagggaccccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggtccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgactggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgctaaaggcgatgctggtccccctggccctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggcgagaaaggatcccctggtgctgatggtcctgctggtgctcctggtactcccgggcctcaaggtattgctggacagcgtggtgtggtcggcctgcctggtcagagaggagagagaggcttccctggtcttcctggcccctctggtgaacctggcaaacaaggtccctctggagcaagtggtgaacgtggtccccctggtcccatgggcccccctggattggctggaccccctggtgaatctggacgtgagggggctcctggtgccgaaggttcccctggacgagacggttctcctggcgccaagggtgaccgtggtgagaccggccccgctggaccccctggtgctcctggtgctcctggtgcccctggccccgttggccctgctggcaagagtggtgatcgtggtgagactggtcctgctggtcccgccggtcctgtcggccctgttggcgcccgtggccccgccggaccccaaggcccccgtggtgacaagggtgagacaggcgaacagggcgacagaggcataaagggtcaccgtggcttctctggcctccagggtccccctggccctcctggctctcctggtgaacaaggtccctctggagcctctggtcctgctggtccccgaggtccccctggctctgctggtgctcctggcaaagatggactcaacggtctccctggccccattgggccccctggtcctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggcagccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggccatccagctgaccttcctgcgcctgatgtccaccgaggcctcccagaacatcacctaccactgcaagaacagcgtggcctacatggaccagcagactggcaacctcaagaaggccctgctcctccagggctccaacgagatcgagatccgcgccgagggcaacagccgcttcacctacagcgtcactgtcgatggctgcacgagtcacaccggagcctggggcaagacagtgattgaatacaaaaccaccaagacctcccgcctgcccatcatcgatgtggcccccttggacgttggtgccccagaccaggaattcggcttcgacgttggccctgtctgcttcctgtaaactccctccatcccaacctggctccctcccacccaaccaactttccccccaacccggaaacagacaagcaacccaaactgaaccccctcaaaagccaaaaaatgggagacaatttcacatggactttggaaaatatttttttcctttgcattcatctctcaaacttagtttttatctttgaccaaccgaacatgaccaaaaaccaaaagtgcattcaaccttaccaaaaaaaaaaaaaaaaaaagaataaataaataactttttaaaaaaggaagcttggtccacttgcttgaagacccatgcgggggtaagtccctttctgcccgttgggcttatgaaaccccaatgctgccctttctgctcctttctccacaccccccttggggcctcccctccactccttcccaaatctgtctccccagaagacacaggaaacaatgtattgtctgcccagcaatcaaaggcaatgctcaaacacccaagtggcccccaccctcagcccgctcctgcccgcccagcacccccaggccctgggggacctggggttctcagactgccaaagaagccttgccatctggcgctcccatggctcttgcaacatctccccttcgtttttgagggggtcatgccgggggagccaccagcccctcactgggttcggaggagagtcaggaagggccacgacaaagcagaaacatcggatttggggaacgcgtgtcaatcccttgtgccgcagggctgggcgggagagactgttctgttccttgtgtaactgtgttgctgaaagactacctcgttcttgtcttgatgtgtcaccggggcaactgcctgggggcggggatgggggcagggtggaagcggctccccattttataccaaaggtgctacatctatgtgatgggtggggtggggagggaatcactggtgctatagaaattgagatgcccccccaggccagcaaatgttcctttttgttcaaagtctatttttattccttgatatttttctttttttttttttttttttgtggatggggacttgtgaatttttctaaaggtgctatttaacatgggaggagagcgtgtgcggctccagcccagcccgctgctcactttccaccctctctccacctgcctctggcttctcaggcctctgctctccgacctctctcctctgaaaccctcctccacagctgcagcccatcctcccggctccctcctagtctgtcctgcgtcctctgtccccgggtttcagagacaacttcccaaagcacaaagcagtttttccccctaggggtgggaggaagcaaaagactctgtacctattttgtatgtgtataataatttgagatgtttttaattattttgattgctggaataaagcatgtggaaatgacccaaacataa

SEQ ID NO: 8 - DNA, 5411 bp, Homo sapiens
  source 1..5411: mol_type = genomic DNA
gtgtcccatagtgtttccaaacttggaaagggcgggggagggcgggaggatgcggagggcggaggtatgcagacaacgagtcagagtttccccttgaaagcctcaaaagtgtccacgtcctcaaaaagaatggaaccaatttaagaagccagccccgtggccacgtcccttcccccattcgctccctcctctgcgcccccgcaggctcctcccagctgtggctgcccgggcccccagccccagccctcccattggtggaggcccttttggaggcaccctagggccagggaaacttttgccgtataaatagggcagatccgggctttattattttagcaccacggcagcaggaggtttcggctaagttggaggtactggccacgactgcatgcccgcgcccgccaggtgatacctccgccggtgacccaggggctctgcgacacaaggagtctgcatgtctaagtgctagacatgctcagctttgtggatacgcggactttgttgctgcttgcagtaaccttatgcctagcaacatgccaatctttacaagaggaaactgtaagaaagggcccagccggagatagaggaccacgtggagaaaggggtccaccaggccccccaggcagagatggtgaagatggtcccacaggccctcctggtccacctggtcctcctggcccccctggtctcggtgggaactttgctgctcagtatgatggaaaaggagttggacttggccctggaccaatgggcttaatgggacctagaggcccacctggtgcagctggagccccaggccctcaaggtttccaaggacctgctggtgagcctggtgaacctggtcaaactggtcctgcaggtgctcgtggtccagctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggacgtgttggtgcccctggcccagctggtgcccgtggcagtgatggaagtgtgggtcccgtgggtcctgctggtcccattgggtctgctggccctccaggcttcccaggtgcccctggccccaagggtgaaattggagctgttggtaacgctggtcctgctggtcccgccggtccccgtggtgaagtgggtcttccaggcctctccggccccgttggacctcctggtaatcctggagcaaacggccttactggtgccaagggtgctgctggccttcccggcgttgctggggctcccggcctccctggaccccgcggtattcctggccctgttggtgctgccggtgctactggtgccagaggacttgttggtgagcctggtccagctggctccaaaggagagagcggtaacaagggtgagcccggctctgctgggccccaaggtcctcctggtcccagtggtgaagaaggaaagagaggccctaatggggaagctggatctgccggccctccaggacctcctgggctgagaggtagtcctggttctcgtggtcttcctggagctgatggcagagctggcgtcatgggccctcctggtagtcgtggtgcaagtggccctgctggagtccgaggacctaatggagatgctggtcgccctggggagcctggtctcatgggacccagaggtcttcctggttcccctggaaatatcggccccgctggaaaagaaggtcctgtcggcctccctggcatcgacggcaggcctggcccaattggcccagctggagcaagaggagagcctggcaacattggattccctggacccaaaggccccactggtgatcctggcaaaaacggtgataaaggtcatgctggtcttgctggtgctcggggtgctccaggtcctgatggaaacaatggtgctcagggacctcctggaccacagggtgttcaaggtggaaaaggtgaacagggtccccctggtcctccaggcttccagggtctgcctggcccctcaggtcccgctggtgaagttggcaaaccaggagaaaggggtctccatggtgagtttggtctccctggtcctgctggtccaagaggggaacgcggtcccccaggtgagagtggtgctgccggtcctactggtcctattggaagccgaggtccttctggacccccagggcctgatggaaacaagggtgaacctggtgtggttggtgctgtgggcactgctggtccatctggtcctagtggactcccaggagagaggggtgctgctggcatacctggaggcaagggagaaaagggtgaacctggtctcagaggtgaaattggtaaccctggcagagatggtgctcgtggtgctcctggtgctgtaggtgcccctggtcctgctggagccacaggtgaccggggcgaagctggggctgctggtcctgctggtcctgctggtcctcggggaagccctggtgaacgtggtgaggtcggtcctgctggccccaatggatttgctggtcctgctggtgctgctggtcaacctggtgctaaaggagaaagaggagccaaagggcctaagggtgaaaacggtgttgttggtcccacaggccccgttggagctgctggcccagctggtccaaatggtccccccggtcctgctggaagtcgtggtgatggaggcccccctggtatgactggtttccctggtgctgctggacggactggtcccccaggaccctctggtatttctggccctcctggtccccctggtcctgctgggaaagaagggcttcgtggtcctcgtggtgaccaaggtccagttggccgaactggagaagtaggtgcagttggtccccctggcttcgctggtgagaagggtccctctggagaggctggtactgctggacctcctggcactccaggtcctcagggtcttcttggtgctcctggtattctgggtctccctggctcgagaggtgaacgtggtctaccaggtgttgctggtgctgtgggtgaacctggtcctcttggcattgccggccctcctggggcccgtggtcctcctggtgctgtgggtagtcctggagtcaacggtgctcctggtgaagctggtcgtgatggcaaccctgggaacgatggtcccccaggtcgcgatggtcaacccggacacaagggagagcgcggttaccctggcaatattggtcccgttggtgctgcaggtgcacctggtcctcatggccccgtgggtcctgctggcaaacatggaaaccgtggtgaaactggtccttctggtcctgttggtcctgctggtgctgttggcccaagaggtcctagtggcccacaaggcattcgtggcgataagggagagcccggtgaaaaggggcccagaggtcttcctggcttaaagggacacaatggattgcaaggtctgcctggtatcgctggtcaccatggtgatcaaggtgctcctggctccgtgggtcctgctggtcctaggggccctgctggtccttctggccctgctggaaaagatggtcgcactggacatcctggtacagttggacctgctggcattcgaggccctcagggtcaccaaggccctgctggcccccctggtccccctggccctcctggacctccaggtgtaagcggtggtggttatgactttggttacgatggagacttctacagggctgaccagcctcgctcagcaccttctctcagacccaaggactatgaagttgatgctactctgaagtctctcaacaaccagattgagacccttcttactcctgaaggctctagaaagaacccagctcgcacatgccgtgacttgagactcagccacccagagtggagcagtggttactactggattgaccctaaccaaggatgcactatggatgctatcaaagtatactgtgatttctctactggcgaaacctgtatccgggcccaacctgaaaacatcccagccaagaactggtataggagctccaaggacaagaaacacgtctggctaggagaaactatcaatgctggcagccagtttgaatataatgtagaaggagtgacttccaaggaaatggctacccaacttgccttcatgcgcctgctggccaactatgcctctcagaacatcacctaccactgcaagaacagcattgcatacatggatgaggagactggcaacctgaaaaaggctgtcattctacagggctctaatgatgttgaacttgttgctgagggcaacagcaggttcacttacactgttcttgtagatggctgctctaaaaagacaaatgaatggggaaagacaatcattgaatacaaaacaaataagccatcacgcctgcccttccttgatattgcacctttggacatcggtggtgctgaccaggaattctttgtggacattggcccagtctgtttcaaataaatgaactcaatctaaattaaaaaagaaagaaatttgaaaaaactttctctttgccatttcttcttcttcttttttaactgaaagctgaatccttccatttcttctgcacatctacttgcttaaattgtgggcaaaagagaaaaagaaggattgatcagagcattgtgcaatacagtttcattaactccttcccccgctcccccaaaaatttgaatttttttttcaacactcttacacctgttatggaaaatgtcaacctttgtaagaaaaccaaaataaaaattgaaaaataaaaaccataaacatttgcaccacttgtggcttttgaatatcttccacagagggaagtttaaaacccaaacttccaaaggtttaaactacctcaaaacactttcccatgagtgtgatccacattgttaggtgctgacctagacagagatgaactgaggtccttgttttgttttgttcataatacaaaggtgctaattaatagtatttcagatacttgaagaatgttgatggtgctagaagaatttgagaagaaatactcctgtattgagttgtatcgtgtggtgtattttttaaaaaatttgatttagcattcatattttccatcttattcccaattaaaagtatgcagattatttgcccaaatcttcttcagattcagcatttgttctttgccagtctcattttcatcttcttccatggttccacagaagctttgtttcttgggcaagcagaaaaattaaattgtacctattttgtatatgtgagatgtttaaataaattgtgaaaaaaatgaaataaagcatgtttggttttccaaaagaacatat

SEQ ID NO: 9 - DNA, 4467 bp, Homo sapiens
  source 1..4467: mol_type = genomic DNA
atggctcacgctcgtgttctcctcctcgctctcgctgttttggcaacagctgctgtggctgtggcttctagttcttcttttgctgattcaaaccctattagacctgttactgatagagcagcttccactttggctcaattgcaagaggagggccaggttgagggccaagatgaggatatccctccaattacatgcgtgcaaaatggcttgcgttaccacgatagggatgtgtggaaacctgaaccttgtcgtatctgtgtgtgtgataacggcaaggtgctctgcgatgatgttatctgcgatgagacaaaaaattgccctggcgctgaagttcctgagggcgagtgttgccctgtgtgccctgatggttccgagtccccaactgatcaggaaactactggcgtggagggcccaaaaggagatactggtccacgtggtcctaggggtccagcaggtcctccaggtagagatggtattccaggccagcctggattgccaggaccaccaggcccacctggcccaccaggacctcctggtcttggtggaaatttcgctccacaactctcttatggctatgatgagaagtcaacaggtggtatttccgttccaggtcctatgggaccatccggaccaagaggtctcccaggtcctccaggtgctcctggacctcaaggctttcaaggacctccaggcgaaccaggagaaccaggcgcttctggaccaatgggcccaaggggaccacctggcccaccaggaaaaaatggcgatgatggcgaagctggaaagcctggtcgtcctggagagagaggtcctcctggcccacagggtgcaagaggcttgccaggaactgctggcttgcctggaatgaagggacataggggcttctccggcctcgatggcgctaagggtgatgctggccctgctggaccaaagggcgagccaggttcccctggagaaaacggtgctcctggacaaatgggtcctcgtggacttccaggagaaaggggtcgtccaggcgctccaggaccagcaggtgctaggggaaacgatggtgcaacaggcgctgctggccctcctggcccaactggtcctgctggccctccaggattcccaggcgcagttggagctaaaggagaagcaggaccacagggccctaggggttctgaaggacctcagggtgttagaggtgaaccaggtcctccaggcccagctggagcagctggtccagcaggaaatccaggtgctgatggtcaacctggagctaagggcgctaatggcgcaccaggtatcgcaggcgcaccaggttttcctggcgctagaggcccaagtggtcctcaaggaccaggtggaccaccaggtccaaaaggcaattctggcgaacctggcgctccaggttctaaaggagatactggtgctaaaggcgaaccaggacctgttggtgttcagggtcctcctggtcctgctggagaagaaggaaaaagaggtgctcgtggagaaccaggaccaactggacttcctggacctcctggtgaacgtggcggacctggctcaaggggtttccctggagctgatggagtggcaggtccaaaaggccctgctggagagagaggttcaccaggtccagctggtcctaagggctcccctggtgaagcaggtagaccaggcgaagcaggattgccaggcgcaaagggattgacaggctctcctggtagtcctggcccagatggaaaaacaggcccaccaggtccagcaggacaagatggacgtccaggcccaccaggtcctcctggagcaaggggacaagctggcgttatgggttttccaggacctaaaggtgctgctggagagccaggaaaggcaggtgaaagaggagttcctggtccaccaggagcagtgggtcctgctggcaaagatggtgaagctggagcacagggccctccaggccctgctggcccagctggcgaacgtggagaacaaggcccagctggtagtccaggatttcaaggattgcctggccctgctggccctccaggagaagcaggaaaacctggagaacaaggagttcctggtgatttgggagcacctggaccttcaggagcacgtggtgaaagaggcttccctggcgagaggggtgttcaaggtccaccaggtccagcaggacctagaggtgctaatggcgctcctggcaacgatggagcaaaaggtgatgctggtgctcctggcgcacctggaagtcagggtgctcctggattgcaaggaatgcctggagagaggggtgctgctggcttgccaggcccaaagggcgataggggtgatgctggaccaaaaggtgctgatggatccccaggaaaagatggagttcgtggtcttactggcccaatcggacctccaggccctgctggcgctccaggtgataagggcgaaagtggcccaagtggacctgctggacctactggtgctagaggtgcacctggtgataggggtgaacctggaccacctggtccagctggttttgctggtcctcctggagctgatggacaacctggcgcaaagggtgaaccaggtgatgctggcgcaaagggagatgctggtccacctggacctgctggtccagcaggcccccctgggccaatcggtaatgttggagcaccaggtgctaagggagctaggggttccgctggtccacctggagcaacaggatttccaggcgctgctggtagagttggcccaccaggcccatccggaaacgcaggccctcctggtcctccaggtcctgctggcaaggagggtggcaaaggaccaaggggcgaaactggccctgctggtagacctggcgaagttggccctcctggaccaccaggtccagcaggagaaaaaggttccccaggagctgatggcccagctggtgctccaggaactccaggccctcaaggtattgctggacagagaggcgttgtgggactccctggtcaaaggggagagagaggatttccaggcttgccaggacctagtggagaacctggaaaacaaggcccatcaggcgctagtggagagcgtggacctcctggccctatgggacctcctggattggctggcccacctggcgaatcaggtcgtgaaggcgcaccaggcgcagaaggatcacctggaagagatggatcccctggtgctaaaggcgatcgtggagaaactggtccagcaggcccaccaggcgcaccaggtgcacctggcgctccaggacctgtgggaccagctggaaaatccggagataggggcgagacaggcccagcaggaccagctggacctgttggccctgctggcgctcgtggaccagcaggacctcaaggaccaaggggagataagggagaaacaggcgaacaaggcgataggggcattaagggtcataggggttttagtggcctccagggtcctcctggcccacctggatcaccaggagaacagggaccatctggtgcttccggcccagctggtccaagaggacctccaggatcagctggtgcacctggaaaagatggtcttaacggtctcccaggaccaatcggccctccaggacctagaggaagaacaggagatgctggccctgttggccctccaggacctcctggtccaccaggtccacctggtcctccatcagctggattcgatttttcatttcttccacagccaccacaagagaaagctcacgatggcggcagatattaccgtgctgatgatgctaacgttgttagggatagagatttggaagtggatacaactttgaaatccctctcccagcaaattgaaaacattagatctccagaaggttcacgtaaaaacccagctagaacatgtcgtgatttgaaaatgtgtcactccgattggaaaagtggtgaatactggattgatccaaatcagggctgtaatctcgatgctatcaaagttttctgtaacatggaaacaggcgaaacatgcgtttatcctactcaaccttccgtggctcagaaaaattggtacatctcaaaaaatcctaaagataagaggcacgtttggttcggtgaaagtatgactgatggatttcaatttgagtacggcggtcaaggtagtgatccagctgatgtggctattcaactcacatttttgcgtcttatgtccacagaggcatcacaaaacatcacttaccactgcaaaaacagtgtggcttatatggatcaacaaacaggaaaccttaagaaggctcttcttttgaagggctcaaacgagattgagattagagcagagggcaactcaaggtttacttattcagttactgttgatggctgcacttcacatactggcgcttggggtaaaacagttatcgagtataagactacaaaaacatcaagactcccaatcattgatgttgctcctctcgatgttggcgctcctgatcaagagttcggttttgatgtgggcccagtttgtttcctc

SEQ ID NO: 10 - DNA, 4167 bp, Homo sapiens
  source 1..4167: mol_type = genomic DNA
atggctcacgctcgtgttctcctcctcgctctcgctgttttggcaacagctgctgtggctgtggcttcaagttctagttttgctgattccaacccaattcgtccagttactgatagagcagcttccactttggctcaattgcttcaagaagaaactgtgaggaagggccctgctggcgataggggccctaggggcgaaaggggtccaccaggacctccaggcagggatggcgaagatggtccaactggccctcctggacctcctggccctccagggccacccggcttgggcggaaacttcgcagctcaatacgatggcaagggtgttggtcttggtcctggtcctatgggcttgatgggacctagaggcccacctggtgctgctggtgctcctggaccacagggttttcagggaccagctggcgagccaggagagccaggccaaacaggaccagctggtgcaaggggacctgctggacctcctggaaaagctggtgaagatggtcacccaggcaaaccaggacgtcctggcgaaagaggtgttgttggaccacaaggcgctaggggatttccaggtacacctggattgccaggttttaagggcattcgtggtcataacggcctcgatggattgaagggacagcctggcgcacctggcgttaagggtgaacctggagcaccaggtgaaaacggtactcctggccagactggtgcaagaggactcccaggtgaaaggggtagagttggtgctcctggacctgctggagctaggggtagtgatggtagtgttggtcctgtgggccctgctggtccaatcggttccgctggcccacctggattcccaggcgctccaggacctaaaggagaaatcggtgctgtgggtaacgcaggtcctactggtccagcaggtcctcgtggagaagtgggattgccaggactttctggtccagtgggccctccaggcaaccctggagctaacggcttgacaggagctaaaggcgcagcaggactccctggagtggctggcgcaccaggattgcctggtccaaggggtatcccaggccctgttggcgcagctggagctactggtgcacgtggacttgttggcgaaccaggccctgctggatcaaaaggcgagtctggaaataagggagaacctggttctgctggacctcaaggtcctcctggaccttctggagaagaaggaaaaaggggaccaaatggcgaggctggatcagcaggtccaccaggaccacctggacttcgtggatcccctggtagtagaggacttccaggcgctgatggtagagcaggcgttatgggaccaccaggaagtagaggagcatccggtccagcaggagttaggggtcctaacggagatgctggtagaccaggtgaaccaggtcttatgggcccaaggggcctcccaggtagtccaggaaatatcggccctgctggaaaagaaggccctgttggacttccaggtattgatggacgtcctggccctattggcccagcaggtgcaagaggagaacctggcaatattggatttccaggaccaaagggtccaacaggcgatcctggaaaaaatggagataagggtcatgctggattggcaggcgcaaggggcgctcctggtccagatggaaacaacggcgcacagggtccacctggccctcagggtgttcaaggcggaaaaggcgaacaaggcccagctggaccaccaggctttcaaggcttgccaggaccaagtggtccagcaggtgaagttggcaagccaggcgagcgtggacttcatggcgagtttggactccctggaccagcaggaccaaggggtgaaagaggccctcctggagagagtggcgctgctggaccaacaggcccaatcggtagtagaggtcctagtggacctccaggcccagatggaaataagggtgaaccaggagttgtgggcgctgttggaacagctggtccttcaggaccatcaggactcccaggcgagagaggcgctgctggcattcctggaggaaaaggtgaaaaaggcgaacctggcctccgtggcgaaatcggaaatcctggacgtgatggtgctcgtggtgcacacggcgctgtgggcgctccaggccctgctggtgctactggtgatagaggagaggctggcgcagctggcccagcaggtcctgctggcccaaggggtagtcctggtgaaagaggcgaagttggacctgctggccctaacggctttgctggccctgctggagcagcaggtcaacctggcgctaaaggtgaaaggggcggaaagggcccaaaaggtgaaaatggcgttgtgggaccaactggtccagtgggcgcagctggacctgctggtccaaatggaccaccaggaccagcaggtagtagaggagatggtggacctccaggaatgacaggttttccaggtgctgctggtagaacaggacctcctggtcctagtggtatttctggtccaccaggaccaccaggtcctgctggaaaagaaggattgaggggtccacgtggtgatcaaggaccagtgggcagaactggtgaagttggcgcagtgggaccacctggttttgctggagaaaagggcccttctggagaggcaggaacagctggtcctcctggtacacctggacctcaaggacttttgggtgcacctggtattctcggattgccaggaagtaggggcgaacgtggacttcctggcgtggcaggagcagttggagaacctggccctctcggaatcgcaggcccaccaggcgcaagaggaccaccaggagctgttggatcaccaggcgtgaatggtgcacctggcgaggctggtcgtgatggaaacccaggaaatgatggcccaccaggaagagatggtcaacctggacacaaaggcgagaggggctacccaggaaatattggcccagttggtgctgctggcgcaccaggcccacacggtccagttggaccagcaggaaaacacggtaatcgtggcgaaacaggcccttcaggcccagtgggacctgctggtgctgttggcccaagaggaccatctggacctcaaggcattagaggcgataagggagagcctggcgaaaaaggacctagaggcttgcctggttttaaaggacacaacggtctccaaggacttccaggtatcgctggtcatcatggagatcagggtgctcctggatcagtgggtccagcaggtcctagaggcccagcaggcccttccggtccagcaggaaaggatggacgtactggccaccctggaactgtgggccctgctggaattagaggtcctcaaggtcatcagggccctgctggccctccaggtccaccaggtcctccaggcccaccaggagtttcaggtggtggttacgattttggttacgatggtgatttttaccgtgctgatcaacctagaagtgctccttctctccgtcctaaagattatgaagttgatgctactttgaaatcacttaacaaccagattgagactcttctcacacctgagggatcaagaaagaatccagcacgtacatgccgtgatctcagacttagtcacccagagtggtcaagtggctattattggattgatcctaatcagggttgtacaatggaggctatcaaagtttactgtgattttccaactggagagacatgtattagggcacaacctgagaacattccagctaaaaattggtatcgttcctctaaagataagaaacatgtttggctcggagagactattaacgctggttctcagttcgagtataatgttgagggcgttacttctaaagagatggcaactcagctcgcttttatgagattgctcgctaactacgcatcccaaaacatcacttatcactgcaaaaattccattgcatatatggatgaggagacaggaaatttgaagaaagcagttattctccaaggtagtaacgatgttgagcttgtggctgagggaaatagtagattcacttacacagttttggtggatggatgctcaaagaaaactaatgagtggggcaagacaatcattgagtacaagacaaataagccttctaggctcccatttctcgatattgcacctcttgatatcggaggagctgatcacgagttttttgttgatatcggacctgtttgttttaag

SEQ ID NO: 11 - DNA, 1633 bp, synthetic construct
  misc_feature 1..1633: note = Synthetic sequence containing the coding regions of the vascular signal sequence of barley gene for Thiol protease aleurain precursor fused to the human Prolyl 4-hydroxylase beta subunit and flanking regions
  source 1..1633: mol_type = other DNA
ctcgagtaaaccatggctcatgctagggttttgcttttggctcttgctgttcttgctactgctgctgttgctgtggcttcttcttcatctttcgctgattctaacccaattaggccagtgactgatagagctgcttctactcttgctcaattggtcgacatggatgctccagaagaggaggatcacgttcttgtgcttaggaagtctaacttcgctgaagctcttgctgctcacaagtaccttcttgtggagttttatgctccttggtgcggacattgcaaagctcttgctccagagtatgctaaggctgctggaaagttgaaggctgagggatctgaaattaggcttgctaaagtggatgctactgaggagtctgatcttgctcaacagtacggagttaggggatacccaactattaagttcttcaggaacggagatactgcttctccaaaggagtatactgctggaagggaggctgatgatattgtgaactggcttaagaagagaactggaccagctgctactactcttccagatggagctgctgctgaatctcttgtggagtcatctgaggtggcagtgattggattcttcaaggatgtggagtctgattctgctaagcagttccttcaagctgctgaggctattgatgatattccattcggaattacttctaactctgatgtgttctctaagtaccagcttgataaggatggagtggtgcttttcaagaaattcgatgagggaaggaacaatttcgagggagaggtgacaaaggagaaccttcttgatttcattaagcacaaccagcttccacttgtgattgagttcactgagcagactgctccaaagattttcggaggagagattaagactcacattcttcttttccttccaaagtctgtgtctgattacgatggaaagttgtctaacttcaagactgctgctgagtctttcaagggaaagattcttttcattttcattgattctgatcacactgataaccagaggattcttgagttcttcggacttaagaaggaagagtgcccagctgttaggcttattactcttgaggaggagatgactaagtacaagccagagtctgaagaacttactgctgagaggattactgagttctgccacagattccttgagggaaagattaagccacaccttatgtctcaagagcttccagaggattgggataagcagccagttaaggtgttggtgggtaaaaacttcgaggatgtggctttcgatgagaagaagaacgtgttcgtggagttctacgcaccttggtgtggtcactgtaagcagcttgctccaatttgggataagttgggagagacttacaaggatcacgagaacattgtgattgctaagatggattctactgctaacgaggtggaggctgttaaggttcactctttcccaactttgaagttcttcccagcttctgctgataggactgtgattgattacaacggagaaaggactcttgatggattcaagaagttccttgagtctggaggacaagatggagctggagatgatgatgatcttgaggatttggaagaagctgaggagccagatatggaggaggatgatgatcagaaggctgtgtgatgagctc

SEQ ID NO: 12 - DNA, 1723 bp, synthetic construct
  misc_feature 1..1723: note = Synthetic sequence containing the coding regions of the vascular signal sequence of barley gene for Thiol protease aleurain precursor fused to the human Prolyl 4-hydroxylase alpha-1 subunit and flanking regions
  source 1..1723: mol_type = other DNA
ctcgagtaaaccatggctcatgctagggttttgcttttggctcttgctgttcttgctactgctgctgttgctgtggcttcttcttcatctttcgctgattctaacccaattaggccagtgactgatagagctgcttctactcttgctcaattggtcgacatgcacccaggattcttcacttctattggacagatgactgatcttattcacactgagaaggatcttgtgacttctcttaaggattacattaaggctgaggaggataagttggagcagattaagaagtgggctgagaagttggataggcttacttctactgctacaaaagatccagagggattcgttggtcatccagtgaacgctttcaagttgatgaagaggcttaacactgagtggagtgagcttgagaaccttgtgcttaaggatatgtctgatggattcatttctaaccttactattcagaggcagtacttcccaaatgatgaggatcaagtgggagctgctaaggctcttcttaggcttcaggatacttacaaccttgatactgatacaatttctaagggaaaccttccaggagttaagcacaagtctttccttactgctgaggattgcttcgagcttggaaaggttgcatacactgaggctgattactaccacactgagctttggatggaacaagctcttaggcaacttgatgagggagagatttctactattgataaggtgtcagtgcttgattacctttcttacgctgtgtaccagcagggtgatcttgataaggctcttttgcttactaagaagttgcttgagcttgatccagaacatcagagggctaacggaaaccttaagtacttcgagtacattatggctaaggaaaaggatgtgaacaagtctgcttctgatgatcagtctgatcaaaagactactccaaagaagaagggagtggctgttgattatcttcctgagaggcagaagtatgagatgttgtgtaggggagagggtattaagatgactccaaggaggcagaagaagttgttctgcaggtatcacgatggaaacaggaacccaaagttcattcttgctccagctaagcaagaagatgagtgggataagccaaggattattaggttccacgatattatttctgatgctgagattgagattgtgaaggatcttgctaagccaagacttaggagggctactatttctaaccctattactggtgatcttgagactgtgcactacaggatttctaagtctgcttggctttctggatacgagaacccagtggtgtctaggattaacatgaggattcaggatcttactggacttgatgtgtctactgctgaggagcttcaagttgctaactacggagttggaggacaatatgagccacacttcgatttcgctaggaaggatgagccagatgcttttaaggagcttggaactggaaacaggattgctacttggcttttctacatgtctgatgtttctgctggaggagctactgttttcccagaagtgggagcttctgtttggccaaagaagggaactgctgtgttctggtacaaccttttcgcttctggagagggagattactctactaggcatgctgcttgcccagttcttgttggaaacaagtgggtgtcaaacaagtggcttcatgagaggggacaagagtttagaaggccatgcactctttctgagcttgagtgatgagctc

SEQ ID NO: 13 - DNA, 2888 bp, synthetic construct
  misc_feature 1..2888: note = Synthetic sequence containing the coding regions of the vascular signal sequence of barley gene for Thiol protease aleurain precursor fused to the human Lysyl hydroxylase 3 and flanking regions
  source 1..2888: mol_type = other DNA
gcgaattcgctagctatcactgaaaagacagcaagacaatggtgtctcgatgcaccagaaccacatctttgcagcagatgtgaagcagccagagtggtccacaagacgcactcagaaaaggcatcttctaccgacacagaaaaagacaaccacagctcatcatccaacatgtagactgtcgttatgcgtcggctgaagataagactgaccccaggccagcactaaagaagaaataatgcaagtggtcctagctccactttagctttaataattatgtttcattattattctctgcttttgctctctatataaagagcttgtattttcatttgaaggcagaggcgaacacacacacagaacctccctgcttacaaaccagatcttaaaccatggctcacgctagggttttgcttcttgctcttgctgttcttgctactgctgctgttgctgtggcttcttcaagttctttcgctgattctaacccaattaggccagtgactgatagagctgcttctactcttgctcaattgagatctatgtctgatagaccaaggggaagggatccagttaatccagagaagttgcttgtgattactgtggctactgctgagactgaaggataccttagattccttaggagtgctgagttcttcaactacactgtgaggactcttggacttggagaagaatggaggggaggagatgttgctagaactgttggaggaggacagaaagtgagatggcttaagaaagagatggagaagtacgctgatagggaggatatgattattatgttcgtggattcttacgatgtgattcttgctggatctccaactgagcttttgaagaaattcgttcagtctggatctaggcttcttttctctgctgagtctttttgttggccagaatggggacttgctgagcaatatccagaagtgggaactggaaagagattccttaactctggaggattcattggattcgctactactattcaccagattgtgaggcagtggaagtacaaggatgacgatgatgatcagcttttctacactaggctttaccttgatccaggacttagggagaagttgtctcttaaccttgatcacaagtctaggattttccagaaccttaacggtgctcttgatgaggttgtgcttaagttcgataggaacagagtgaggattaggaacgtggcttacgatactcttcctattgtggtgcatggaaacggaccaacaaaactccagcttaactaccttggaaactacgttccaaacggatggactccagaaggaggatgtggattctgcaatcaggataggagaactcttccaggaggacaaccaccaccaagagttttccttgctgtgttcgttgaacagccaactccattccttccaagattccttcagaggcttcttcttttggattacccaccagatagggtgacacttttccttcacaacaacgaggttttccacgagccacacattgctgattcttggccacagcttcaggatcatttctctgctgtgaagttggttggtccagaagaagctctttctccaggagaagctagggatatggctatggatttgtgcaggcaggatccagagtgcgagttctacttctctcttgatgctgatgctgtgcttactaaccttcagactcttaggattcttattgaggagaacaggaaagtgattgctccaatgctttctaggcacggaaagttgtggtctaatttctggggtgctctttctcctgatgagtactacgctagatcagaggactacgtggagcttgttcagagaaagagagtgggagtttggaacgttccttatatttctcaggcttacgtgattaggggagatactcttaggatggagcttccacagagggatgttttctctggatctgatactgatccagatatggctttctgcaagtctttcagggataagggaattttccttcacctttctaaccagcatgagttcggaagattgcttgctacttcaagatacgatactgagcaccttcatcctgatctttggcagattttcgataacccagtggattggaaggagcagtacattcacgagaactactctagggctcttgaaggagaaggaattgtggagcaaccatgcccagatgtttactggttcccacttctttctgagcaaatgtgcgatgagcttgttgctgagatggagcattacggacaatggagtggaggtagacatgaggattctaggcttgctggaggatacgagaacgttccaactgtggatattcacatgaagcaagtgggatacgaggatcaatggcttcagcttcttaggacttatgtgggaccaatgactgagtctcttttcccaggataccacactaaggctagggctgttatgaacttcgttgtgaggtatcgtccagatgagcaaccatctcttaggccacaccacgattcttctactttcactcttaacgtggctcttaaccacaagggacttgattatgagggaggaggatgccgtttccttagatacgattgcgtgatttcttcaccaagaaagggatgggctcttcttcatccaggaaggcttactcattaccacgagggacttccaactacttggggaactagatatattatggtgtctttcgtggatccatgactgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccagaattcgcg

SEQ ID NO: 14 - protein, 45 aa, synthetic construct
  REGION 1..45: note = Vacuolar targeting sequence of the thiol protease aleurain precursor (NCBI accession P05167 GI:113603)
MAHARVLLLALAVLATAAVAVASSSSFADSNPIRPVTDRAASTLA

SEQ ID NO: 15 - protein, 1469 aa, Homo sapiens
MAHARVLLLALAVLATAAVAVASSSSFADSNPIRPVTDRAASTLAQLQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPAGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKTSRLPIIDVAPLDVGAPDQEFGFDVGPVCFL

SEQ ID NO: 16 - DNA, 4416 bp, Homo sapiens
  source 1..4416: mol_type = genomic DNA
atggctcacgctcgtgttctcctcctcgctctcgctgttttggcaacagctgctgtggctgtggcttctagttcttcttttgctgattcaaaccctattagacctgttactgatagagcagcttccactttggctcaattgcaagaggagggccaggttgagggccaagatgaggatatccctccaattacatgcgtgcaaaatggcttgcgttaccacgatagggatgtgtggaaacctgaaccttgtcgtatctgtgtgtgtgataacggcaaggtgctctgcgatgatgttatctgcgatgagacaaaaaattgccctggcgctgaagttcctgagggcgagtgttgccctgtgtgccctgatggttccgagtccccaactgatcaggaaactactggcgtggagggcccaaaaggagatactggtccacgtggtcctaggggtccagcaggtcctccaggtagagatggtattccaggccagcctggattgccaggaccaccaggcccacctggcccaccaggacctcctggtcttggtggaaatttcgctccacaactctcttatggctatgatgagaagtcaacaggtggtatttccgttccaggtcctatgggaccatccggaccaagaggtctcccaggtcctccaggtgctcctggacctcaaggctttcaaggacctccaggcgaaccaggagaaccaggcgcttctggaccaatgggcccaaggggaccacctggcccaccaggaaaaaatggcgatgatggcgaagctggaaagcctggtcgtcctggagagagaggtcctcctggcccacagggtgcaagaggcttgccaggaactgctggcttgcctggaatgaagggacataggggcttctccggcctcgatggcgctaagggtgatgctggccctgctggaccaaagggcgagccaggttcccctggagaaaacggtgctcctggacaaatgggtcctcgtggacttccaggagaaaggggtcgtccaggcgctccaggaccagcaggtgctaggggaaacgatggtgcaacaggcgctgctggccctcctggcccaactggtcctgctggccctccaggattcccaggcgcagttggagctaaaggagaagcaggaccacagggccctaggggttctgaaggacctcagggtgttagaggtgaaccaggtcctccaggcccagctggagcagctggtccagcaggaaatccaggtgctgatggtcaacctggagctaagggcgctaatggcgcaccaggtatcgcaggcgcaccaggttttcctggcgctagaggcccaagtggtcctcaaggaccaggtggaccaccaggtccaaaaggcaattctggcgaacctggcgctccaggttctaaaggagatactggtgctaaaggcgaaccaggacctgttggtgttcagggtcctcctggtcctgctggagaagaaggaaaaagaggtgctcgtggagaaccaggaccaactggacttcctggacctcctggtgaacgtggcggacctggctcaaggggtttccctggagctgatggagtggcaggtccaaaaggccctgctggagagagaggttcaccaggtccagctggtcctaagggctcccctggtgaagcaggtagaccaggcgaagcaggattgccaggcgcaaagggattgacaggctctcctggtagtcctggcccagatggaaaaacaggcccaccaggtccagcaggacaagatggacgtccaggcccaccaggtcctcctggagcaaggggacaagctggcgttatgggttttccaggacctaaaggtgctgctggagagccaggaaaggcaggtgaaagaggagttcctggtccaccaggagcagtgggtcctgctggcaaagatggtgaagctggagcacagggccctccaggccctgctggcccagctggcgaacgtggagaacaaggcccagctggtagtccaggatttcaaggattgcctggccctgctggccctccaggagaagcaggaaaacctggagaacaaggagttcctggtgatttgggagcacctggaccttcaggagcacgtggtgaaagaggcttccctggcgagaggggtgttcaaggtccaccaggtccagcaggacctagaggtgctaatggcgctcctggcaacgatggagcaaaaggtgatgctggtgctcctggcgcacctggaagtcagggtgctcctggattgcaaggaatgcctggagagaggggtgctgctggcttgccaggcccaaagggcgataggggtgatgctggaccaaaaggtgctgatggatccccaggaaaagatggagttcgtggtcttactggcccaatcggacctccaggccctgctggcgctccaggtgataagggcgaaagtggcccaagtggacctgctggacctactggtgctagaggtgcacctggtgataggggtgaacctggaccacctggtccagctggttttgctggtcctcctggagctgatggacaacctggcgcaaagggtgaaccaggtgatgctggcgcaaagggagatgctggtccacctggacctgctggtccagcaggcccccctgggccaatcggtaatgttggagcaccaggtgctaagggagctaggggttccgctggtccacctggagcaacaggatttccaggcgctgctggtagagttggcccaccaggcccatccggaaacgcaggccctcctggtcctccaggtcctgctggcaaggagggtggcaaaggaccaaggggcgaaactggccctgctggtagacctggcgaagttggccctcctggaccaccaggtccagcaggagaaaaaggtggacagagaggcgttgtgggactccctggtcaaaggggagagagaggatttccaggcttgccaggacctagtggagaacctggaaaacaaggcccatcaggcgctagtggagagcgtggacctcctggccctatgggacctcctggattggctggcccacctggcgaatcaggtcgtgaaggcgcaccaggcgcagaaggatcacctggaagagatggatcccctggtgctaaaggcgatcgtggagaaactggtccagcaggcccaccaggcgcaccaggtgcacctggcgctccaggacctgtgggaccagctggaaaatccggagataggggcgagacaggcccagcaggaccagctggacctgttggccctgctggcgctcgtggaccagcaggacctcaaggaccaaggggagataagggagaaacaggcgaacaaggcgataggggcattaagggtcataggggttttagtggcctccagggtcctcctggcccacctggatcaccaggagaacagggaccatctggtgcttccggcccagctggtccaagaggacctccaggatcagctggtgcacctggaaaagatggtcttaacggtctcccaggaccaatcggccctccaggacctagaggaagaacaggagatgctggccctgttggccctccaggacctcctggtccaccaggtccacctggtcctccatcagctggattcgatttttcatttcttccacagccaccacaagagaaagctcacgatggcggcagatattaccgtgctgatgatgctaacgttgttagggatagagatttggaagtggatacaactttgaaatccctctcccagcaaattgaaaacattagatctccagaaggttcacgtaaaaacccagctagaacatgtcgtgatttgaaaatgtgtcactccgattggaaaagtggtgaatactggattgatccaaatcagggctgtaatctcgatgctatcaaagttttctgtaacatggaaacaggcgaaacatgcgtttatcctactcaaccttccgtggctcagaaaaattggtacatctcaaaaaatcctaaagataagaggcacgtttggttcggtgaaagtatgactgatggatttcaatttgagtacggcggtcaaggtagtgatccagctgatgtggctattcaactcacatttttgcgtcttatgtccacagaggcatcacaaaacatcacttaccactgcaaaaacagtgtggcttatatggatcaacaaacaggaaaccttaagaaggctcttcttttgaagggctcaaacgagattgagattagagcagagggcaactcaaggtttacttattcagttactgttgatggctgcacttcacatactggcgcttggggtaaaacagttatcgagtataagactacaaaaacatcaagactcccaatcattgatgttgctcctctcgatgttggcgctcctgatcaagagttcggttttgatgtgggcccagtttgtttcctctaatgagct

(19) United States
(12) Patent Application Publication
(10) Pub. No.: US 2025/0032668 A1
(43) Pub. Date: Jan. 30, 2025

(54) ADDITIVE MANUFACTURING USING RECOMBINANT COLLAGEN-CONTAINING FORMULATION

(71) Applicant: CollPlant Ltd., Rehovot (IL)

(72) Inventors: Oded SHOSEYOV, Shoham (IL); Nadav ORR, Mazkeret Batya (IL); Jasmine SEROR MAKNOUZ, Tel-Aviv (IL); Revital ZARKA, Mazkeret Batya (IL)

(73) Assignee: CollPlant Ltd., Rehovot (IL)

(21) Appl. No.: 18/914,347

(22) Filed: Oct. 14, 2024

Related U.S. Application Data
(62) Division of application No. 16/620,556, filed on Dec. 9, 2019, now Pat. No. 12,115,276.
(60) Provisional application No. 62/517,179, filed on Jun. 9, 2017.

(51) Int. Cl. A61L 27/24; B29C 64/106; B33Y 10/00; B33Y 70/00

(52) U.S. Cl. CPC A61L 27/24; B29C 64/106; B33Y 70/00; B33Y 10/00

(57) ABSTRACT

Compositions containing curable recombinant human collagen, and kits comprising same, which are usable in preparing modeling material formulations for additive manufacturing (e.g., 3D bioprinting) of 3D objects are provided. Methods utilizing such modeling material formulations in additive manufacturing of 3D objects having a collagen-based material in at least a portion thereof, are also provided. The formulations feature a desired viscosity at a temperature higher than 10° C. (e.g., room temperature or 37° C.) and allow performing the additive manufacturing without cooling the system or a part thereof.

### RELATED APPLICATION

This application is a division of U.S. patent application Ser. No. 16/620,556, filed on Dec. 9, 2019, which is a National Phase of PCT Patent Application No. PCT/IL2018/050627 having International filing date of Jun. 8, 2018, which claims the benefit of priority under 35 USC § 119(e) of U.S. Provisional Patent Application No. 62/517,179 filed on Jul. 9, 2017[[ ]]. The contents of the above applications are all incorporated by reference as if fully set forth herein in their entirety.

### SEQUENCE LISTING STATEMENT

The XML file, entitled 101596SequenceListing.xml, created on Oct. 5, 2024, comprising 57,344 bytes, submitted concurrently with the filing of this application is incorporated herein by reference. The sequence listing submitted herewith is identical to the sequence listing forming part of the international application.

### FIELD AND BACKGROUND OF THE INVENTION

The present invention, in some embodiments thereof, relates to additive manufacturing and, more particularly, but not exclusively, to three-dimensional (3D) bioprinting of 3D objects using a collagen-based building material.

Collagen comprises the main component of connective tissue and is the most abundant protein in mammals, comprising approximately 30% of the proteins found in the body. Collagen serves as the predominant component and primary structural-mechanical determinant of most tissue extracellular matrix (ECM) [see, for example, Kadler K. Birth Defects Res C Embryo Today. 2004; 72:1-11; Kadler K E, Baldock C, Bella J, Boot-Handford R P. J Cell Sci. 2007; 120:1955-1958.; Kreger S T. Biopolymers. 2010 93(8): 690-707].

Due to its unique characteristics and diverse profile in human body functions, collagen is frequently selected from a variety of biocompatible materials for use in tissue repair to support structural integrity, induce cellular infiltration and promote tissue regeneration. Among the 5 major collagen types, Type I collagen is the most abundant form of in the human body. Collagen's unique properties make it a favorite choice for regenerative medicine products.

Additive manufacturing (AM) is generally a process in which a three-dimensional (3D) object is manufactured utilizing a computer model of the objects. The basic operation of any AM system consists of slicing a three-dimensional computer model into thin cross sections, translating the result into two-dimensional position data and feeding the data to control equipment which manufacture a three-dimensional structure in a layerwise manner.

Various AM technologies exist, amongst which are stereolithography, digital light processing (DLP), and three-dimensional (3D) printing such as 3D inkjet printing. Such techniques are generally performed by layer by layer deposition and hardening (e.g., solidification) of one or more building materials, which typically include photopolymerizable (photocurable) materials.

Stereolithography, for example, is an additive manufacturing process which employs a liquid ultraviolet (UV)-curable building material and a UV laser. In such a process, for each dispensed layer of the building material, the laser beam traces a cross-section of the part pattern on the surface of the dispensed liquid building material. Exposure to the UV laser light cures and solidifies the pattern traced on the building material and joins it to the layer below. After being built, the formed parts are immersed in a chemical bath in order to be cleaned of excess building material and are subsequently cured in an UV oven.

In three-dimensional printing processes, for example, a building material is dispensed from a dispensing head having a set of nozzles to deposit layers on a supporting structure. Depending on the building material, the layers may then be cured or solidify using a suitable device.

The building materials may include modeling material formulation(s) and support material formulation(s), which form, upon hardening, the object and the temporary support constructions supporting the object as it is being built, respectively.

The modeling material formulation(s) is/are deposited to produce the desired object and the support material formulation(s) is/are used, with or without modeling material elements, to provide support structures for specific areas of the object during building and assure adequate vertical placement of subsequent object layers, e.g., in cases where objects include overhanging features or shapes such as curved geometries, negative angles, voids, and so on.

Both the modeling and support materials are preferably liquid at the working temperature at which they are dispensed, and subsequently hardened, typically upon exposure to hardening or curing condition such as curing energy (e.g., UV curing), to form the required layer shape. After printing completion, support structures, if present, are removed to reveal the final shape of the fabricated 3D object. The hardening (curing) of the dispensed materials typically involves polymerization (e.g., photopolymerization) and/or crosslinking (e.g., photocrosslinking).

Additive manufacturing has been first used in biological applications for forming three-dimensional sacrificial resin molds in which 3D scaffolds from biological materials were created.

3D bioprinting is an additive manufacturing methodology which uses biological materials, optionally in combination with chemicals and/or cells, that are printed layer-by-layer with a precise positioning and a tight control of functional components placement to create a 3D structure.

Three dimensional (3D) bioprinting is gaining momentum in many medicinal applications, especially in regenerative medicine, to address the need for complex scaffolds, tissues and organs suitable for transplantation.

Inherent to 3D printing in general is that the mechanical properties of the printing media (the dispensed building material) are very different from the post-printed cured (hardened) material.

To allow tight control on the curing (e.g., polymerization) after printing, the building material commonly includes polymerizable (e.g., photopolymerizable) moieties or groups that polymerize (e.g., by chain elongation and/or cross-linking) upon being dispensed, to preserve the geometric shape and provide the necessary physical properties of the final product.

Different technologies have been developed for 3D bioprinting, including 3D Inkjet printing, Extrusion printing, Laser-assisted printing and Projection stereolithography [see, for example, Murphy S V, Atala A, Nature Biotechnology. 2014 32(8).; Miller J S, Burdick J. ACS Biomater. Sci. Eng. 2016, 2, 1658-1661]. Each technology has its different requirements for the dispensed building material (also referred to herein as printing media), which is derived from the specific application mechanism and the curing/gelation process required to maintain the 3D structure of the scaffold post printing.

For all technologies, the most important parameter determining the accuracy and efficiency of the printing is the static and dynamic physical properties of the dispensed building material, including viscosity, shear thinning and thixotrophic properties. The static and dynamic properties of the building material are important not only for the printing technology but also when considering cell-laden printing, i.e. including cells in the building material dispensed during printing. In this case, the shearing forces applied to the building material during printing (dispensing) have a significant effect on the survival of the cells. Therefore, it is desirable to have good control on the specific properties of the printing media over a wide range of conditions, i.e. concentration, temperature, ionic strength and pH.

Type I collagen is considered a perfect candidate for use as a major component of a building material in 3D-bioprinting.

Collagen methacrylate can be used as a rapidly self-assembling type I collagen to form cross-linked hydrogels for tissue engineering [see, for example, Isaacson et al., Experimental Eye Research 173, 188-193 (2018)]. It has been used with mesenchymal stem cells [Kathryn E. Drzewiecki et al., A thermoreversible, photocrosslinkable collagen bio-ink for free-form fabrication of scaffolds for regenerative medicine, TECHNOLOGY (2017)], fibroblasts, adipose derived stem cells, epithelial cells, and many more. Collagen methacrylate is useful for forming scaffolds with varying degree of stiffness, by altering collagen concentration or the curing conditions (e.g., intensity and duration of irradiation).

Collagen methacrylate extracted from tissues has been extensively characterized for its usefulness in 3D-bioprinting (extrusion, inkjet, and photolithographic [Drzewiecki, K. E. et al. Langmuir 30, 11204-11211 (2014); Gaudet, I. D. & Shreiber, D. I. Biointerphases 7, 25 (2012)].

Despite the significant advantages offered by this natural polymer, a number of factors hinder its use for 3D bioprinting. The use of tissue extracted collagen for this purpose is limited due to its sensitivity to temperature and ionic strength, which leads to spontaneous gel formation at temperatures higher than 20° C., under physiological conditions [see, for example, PureCol, Advanced BioMatrix, Inc.]. The typical temperature-dependent formation of gel of tissue extracted-collagens hampers significantly the precise fluidity during printing. Keeping the printing media at low temperature until application is a possible solution for this phenomena but implies a serious technical limitation. Another solution is the use of gelatin, the denatured form of collagen which does not become gel-like under these conditions. However, gelatin lacks the genuine tissue and cell interactions of native collagen and thus crucial biological functions are lost.

The present assignee has developed a technology that allows the purification of naïve human Type I collagen (rhCollagen) by introducing into tobacco plants, five human genes encoding heterotrimeric type I collagen [see, for example, Stein H. (2009) Biomacromolecules; 10:2640-5]. The protein is purified to homogeneity through a cost-effective industrial process taking advantage of collagen's unique properties. See also WO 2006/035442, WO 2009/053985, WO 2011/064773, WO 2013/093921, WO 2014/147622, and patents and patent applications deriving therefrom, all of which are incorporated by reference as if fully set forth herein.

Additional background art includes U.S. patent application Ser. No. 15/867,783.

### SUMMARY OF THE INVENTION

According to an aspect of some embodiments of the present invention there is provided a formulation for use, or usable, in additive manufacturing of a three-dimensional object that comprises, in at least a portion thereof, a collagen-based material, the formulation comprising a recombinant human collagen featuring at least one curable group.

According to some of any of the embodiments of the present invention, the at least one curable group is a photocurable group.

According to some of any of the embodiments of the present invention, the recombinant human collagen is a recombinant human Type I collagen.

According to some of any of the embodiments of the present invention, the recombinant human collagen is a plant-derived recombinant collagen.

According to some of any of the embodiments of the present invention, the formulation further comprises and an aqueous carrier.

According to some of any of the embodiments of the present invention, the formulation features a pH that ranges from about 6 to about 8.

According to some of any of the embodiments of the present invention, the aqueous carrier comprises a phosphate buffer.

According to some of any of the embodiments of the present invention, the aqueous carrier comprises an acid.

According to some of any of the embodiments of the present invention, the aqueous carrier comprises a culturing medium.

According to some of any of the embodiments of the present invention, a concentration of the recombinant human collagen in the modeling material formulation ranges from 0.5 mg/mL to 50 mg/mL, or from 0.5 mg/mL to 20 mg/mL.

According to some of any of the embodiments of the present invention, the formulation further comprises at least one curable material other than the recombinant human collagen featuring the curable group.

According to some of any of the embodiments of the present invention, a weight ratio of the recombinant human collagen featuring the curable group and the at least one curable material ranges from 10:1 to 1:2, or from 10:1 to 1:1, or from 5:1 to 2:1.

According to some of any of the embodiments of the present invention, the formulation further comprises an agent that modifies a mechanical and/or rheological and/or physical property of the formulation and/or of a respective portion of the object.

According to some of any of the embodiments of the present invention, the formulation further comprises a biological material other than the human recombinant collagen.

According to some of any of the embodiments of the present invention, the formulation is a shear-thinning formulation.

According to some of any of the embodiments of the present invention, the formulation is a thermal-thinning formulation.

According to some of any of the embodiments of the present invention, the formulation features a viscosity of no more than 2,000 centipoises, or no more than 1,500 centipoises, at a zero shear strain rate, at 37° C., wherein a concentration of the recombinant human collagen featuring a curable group is at least 3 mg/mL.

According to some of any of the embodiments of the present invention, the modeling material formulation features a viscosity of no more than 2000 centipoises, or no more than 1,500 centipoises, at a shear strain rate of 5 1/sec, at room temperature, wherein a concentration of the recombinant human collagen featuring a curable group is at least 3 mg/mL.

According to some of any of the embodiments of the present invention, the formulation features, when hardened, storage modulus (G′) of at least 1,000 Pa.

According to some of any of the embodiments of the present invention, the formulation features, when hardened, an increase of at least 10-folds of its storage modulus (G′).

According to an aspect of some embodiments of the present invention there is provided a process or a method of additive manufacturing of a three-dimensional object featuring, in at least a portion thereof, a collagen-based material, the process comprising dispensing at least one modeling material formulation to sequentially form a plurality of layers in a configured pattern corresponding to a shape of the object, wherein for at least a portion of the layers, the dispensing is of a modeling material formulation that comprises a recombinant human collagen featuring at least one curable group, as described herein in any of the respective embodiments and any combination thereof, thereby manufacturing the three-dimensional object.

According to some of any of the embodiments described herein, the process further comprises exposing the portion of the layers to a curing condition suitable for hardening the recombinant human collagen featuring the at least one curable group.

According to some of any of the embodiments described herein, the at least one curable group is a photocurable group.

According to some of any of the embodiments described herein, the process further comprises exposing the portion of the layers to irradiation.

According to some of any of the embodiments described herein, the recombinant human collagen is a recombinant human Type I collagen.

According to some of any of the embodiments described herein, for at least a portion of the layers, the dispensing is further of a modeling material formulation that comprises at least one curable material other than the recombinant human collagen featuring the curable group.

According to some of any of the embodiments described herein, the dispensing is of a modeling material formulation that comprises the recombinant human collagen featuring the curable group and the at least one curable material.

According to some of any of the embodiments described herein, a weight ratio of the recombinant human collagen featuring the curable group and the at least one curable material ranges from 10:1 to 1:2, or from 10:1 to 1:1, or from 5:1 to 2:1.

According to some of any of the embodiments described herein, the at least one curable material comprises a hyaluronic acid featuring at least one curable group.

According to some of any of the embodiments described herein, the modeling material formulation further comprises an agent that modifies a mechanical and/or rheological and/or physical property of the formulation and/or of a respective portion of the object.

According to some of any of the embodiments described herein, for at least a portion of the layers, the dispensing is further of a modeling material formulation that comprises an agent that modifies a mechanical and/or rheological and/or physical property of the formulation and/or of a respective portion of the object.

According to some of any of the embodiments described herein, the agent is a thixotropic agent.

According to some of any of the embodiments described herein, the agent is a gel-forming agent.

According to some of any of the embodiments described herein, the modeling material formulation further comprises a biological material other than the human recombinant collagen.

According to some of any of the embodiments described herein, for at least a portion of the layers, the dispensing is further of a modeling material formulation that comprises a biological material other than the human recombinant collagen.

According to some of any of the embodiments described herein, the modeling material formulation that comprises the human recombinant collagen further comprises the biological material.

According to some of any of the embodiments of the present invention, the dispensing is at a temperature of at least 10° C., or of at least 20° C., or of 37° C.

According to an aspect of some embodiments of the present invention there is provided a three-dimensional biological object obtainable by the process of AM as described herein in any of the respective embodiments and any combination thereof.

According to some of any of the embodiments of the present invention, the object further comprises a biological material other than the collagen-based material in or on at least a portion thereof.

According to some of any of the embodiments of the present invention, the object is for use, or is usable in, repairing a damaged tissue.

According to some of any of the embodiments of the present invention, the object is an artificial tissue or organ, or is usable, or for use, in constructing an artificial organ or tissue.

According to an aspect of some embodiments of the present invention there is provided a composition comprising a recombinant human collagen featuring at least one curable group, as described herein in any of the respective embodiments and any combination thereof.

According to some of any of the embodiments of the present invention, the composition is for use, or is usable, in preparing a modeling material formulation usable in additive manufacturing of a three-dimensional object that comprises, in at least a portion thereof, a collagen-based material.

According to some of any of the embodiments of the present invention, the composition further comprises an aqueous solution of the recombinant human collagen featuring at least one curable group.

According to an aspect of some embodiments of the present invention there is provided a kit comprising the composition described herein packaged therein, the kit being identified for use in preparing a modeling material formulation usable in additive manufacturing of a three-dimensional object that comprises, in at least a portion thereof, a collagen-based material.

According to some of any of the embodiments of the present invention, the modeling material formulation further comprises an aqueous carrier, and the kit further comprises the aqueous carrier or instructions to prepare the formulation with the aqueous carrier.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof.

Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents ¹H nuclear magnetic resonance (NMR) spectra of rhCollagen (top) and of rhCollagen methacrylate (bottom), as recorded in quantitative analysis using 500 MHz spectrometer. The signals of the methyl function and acrylic protons of the methacrylate introduced in rhCollagen methacrylate are indicated as (b) and (c), respectively, and the lysine methylene signals of the rhCollagen as indicated as (a).

FIG. 2 presents comparative plots showing the viscosity as a function of shear rate, of a solution of 2.7 mg/mL bovine collagen in DPBS (solid line), and of a solution of 2.79 mg/mL rhCollagen in DPBS (dashed line), as measured at 4° C. and at 37° C.

FIG. 3 presents comparative plots showing the viscosity as a function of shear rate, of a solution of 3.4 mg/mL rhCollagen in FB, as measured at 4° C. and at 37° C., demonstrating an identical shear thinning behavior at both temperatures.

FIG. 4 presents comparative plots showing the viscosity as a function of shear rate, of a solution of 10 mg/mL rhCollagen-MA in DPBS as measured at 4° C. and at 37° C., demonstrating a shear thinning behavior at both temperatures.

FIG. 5 presents comparative plots showing the viscosity as function of temperature increase, of a solution of 10 mg/ml rhCollagen-MA in 20 mM acetic acid (white circles) and an equivalent solution of bovine collagen methacrylate ink (bovine type I collagen methacrylate) (black circles), measured in a rheometer using rotational temperature steps CR mode with shear rate of 5 [1/sec].

FIGS. 6A-B are bar graphs presenting the storage and loss moduli and tan phase shift angle (tan delta) of rhCollagen-MA formulation at different concentrations before (FIG. 6A) and after (FIG. 6B) photocuring.

FIG. 7 is a bar graph showing the G′ and G″ values of cured objects prepared from various formulations of fibrillar rhCollagen-MA, at 37° C., recorded in frequency sweep test and plotted at 1 Hz.

FIG. 8 presents plots showing G′ and complexed viscosity values measured over time of cured objects prepared from a solution of 15 mg/ml rhCollagenn-MA in 10 mM HCl, as recorded in a rheometer using Oscillation with 7% strain and 5 rad/sec angular frequency.

FIG. 9 presents comparative plots showing the viscosity as a function of shear rate of a solution containing rhCollagen-MA, rhCollagen-MA+hyaluronic acid (HA), or rhCollagen-MA and hyaluronic acid-MA (HAMA) in DMEM.

FIG. 10 presents comparative plots showing the viscosity as a function of shear rate of a solution of 5 mg/ml rhCollagen-MA (colMA), and of solutions of 5 mg/ml rhCollagen-MA and Polyvinyl alcohol methacrylate (PVAMA), at rhCollagen-MA:PVAMA weight ratio of 5:1, 2:1 or 1:2. All solutions are in 0.1 M PB+11.3 mM NaCl, pH 7.4.

FIG. 11 presents comparative plots showing the viscosity as a function of shear rate of a solution of 5 mg/ml rhCollagen-MA (colMA) and of solutions of 5 mg/ml rhCollagen-MA and HAMA at rhCollagen-MA:HAMA weight ratio of 5:1 or 2. All solutions are in 0.1 MPB+11.3 mM NaCl pH 7.4.

FIG. 12 presents comparative plots showing the viscosity as a function of shear rate of a solution of 5 mg/ml rhCollagen-MA (colMA) and of solutions of 5 mg/ml rhCollagen-MA and TiO₂ at rhCollagen-MA:TiO₂ weight ratio of 5:1, 2:1 (red dashed curve), or 1:2. All solutions are in 0.1 MPB+11.3 mM NaCl pH 7.4.

FIG. 13 presents comparative plots showing the viscosity as a function of shear rate of a solution of 5 mg/ml rhCollagen-MA (colMA) and of solutions of 5 mg/ml rhCollagen-MA and oxidized cellulose at rhCollagen-MA:oxidized cellulose weight ratio of 5:1, 2:1, or 1:2. All solutions are in 0.1 MPB+11.3 mM NaCl pH 7.4.

FIG. 14 presents photographs of cured objects (scaffolds) obtained from formulations containing rhCollagen-MA and various additives at rhCollagen-MA:additive weight ratio of 2:1.

FIG. 15 presents plots showing the viscosity as a function of shear rate of solutions containing 10 mg/ml rhCollagen-MA in phosphate buffer 0.1 M/NaCl 0.016 M (light grey) and in 10 mM HCl (dark grey) solutions before curing, as recorded in the rheometer using Rotational CR mode at R.T., demonstrating a more pronounced shear thinning behavior of the PBS solution.

FIG. 16 is a bar graph showing G′ values of objects obtained upon curing solutions containing 10 mg/ml rhCollagen-MA in phosphate buffer 0.1 M/NaCl 0.016 M and in 10 mM HCl, as recorded in the rheometer using Oscillation CR mode at 37° C.

FIG. 17 presents a photograph of a scaffold prepared from a solution of rhCollagen-MA integrated into a mouse skin patch (ex-vivo) following illumination with a white LED torch through the skin. The skin patch was illuminated on its external side, while the rhCollagen-MA solution was dispensed in a mold underneath the skin FIG. 18 is a bar graph showing the proliferation of normal human dermal fibroblasts seeded in scaffolds made of a solution containing rhCollagen-MA and calcium phosphate particles.

### DESCRIPTION OF SPECIFIC EMBODIMENTS OF THE INVENTION

The present invention, in some embodiments thereof, relates to additive manufacturing and, more particularly, but not exclusively, to three-dimensional (3D) bioprinting of 3D objects using a collagen-based building material.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have surprisingly uncovered that recombinant human collagen (rhCollagen) is much more tolerant to temperature and ionic strength conditions than tissue extracted-collagen. The present inventors have uncovered that an aqueous solution of rhCollagen does not form gels at temperatures above 20° C., and even at 37° C., and that its viscosity is minimally affected at varying conditions, for example, at acidic conditions, or at a salt concentration above 50 mM.

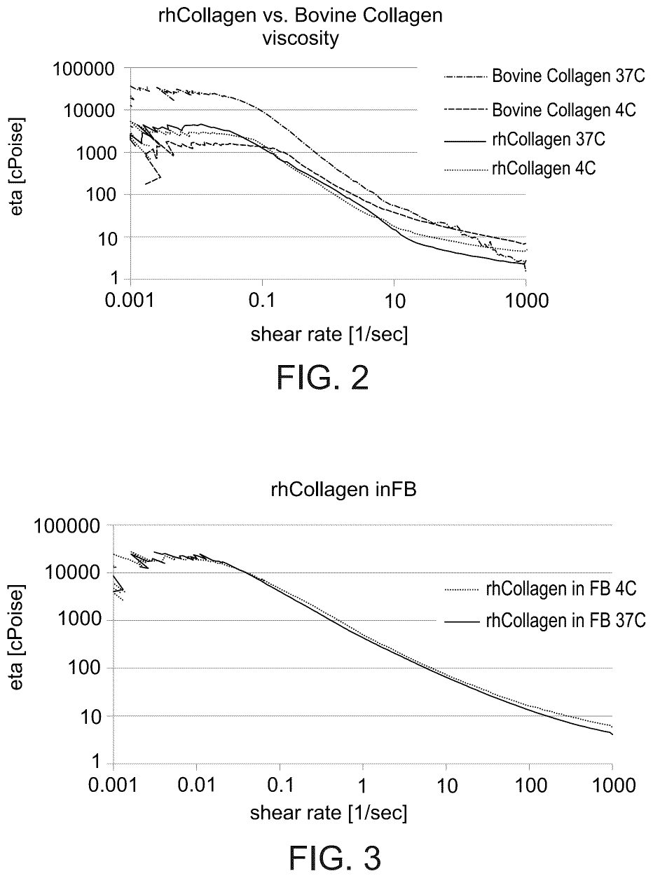

FIGS. 2 and 3 demonstrate the similar viscosity profile at increasing shear rate of an rhCollagen solution at different temperatures, and FIG. 2 further demonstrates the different viscosity profile observed at different temperatures for an aqueous solution of a tissue-extracted collagen, and particularly the substantially higher viscosity of the tissue-extracted collagen solution at low shear rates.

The present inventors have realized that these properties of rhCollagen can be advantageously utilized in additive manufacturing processes (e.g., 3D-bioprinting), by allowing performing such processes at conditions that are compatible with biological additives (e.g., physiological temperature and ionic strength, lower applied shear force), while avoiding the need to cool the additive manufacturing system, and while having a better control on the fluidity of the modeling material formulation.

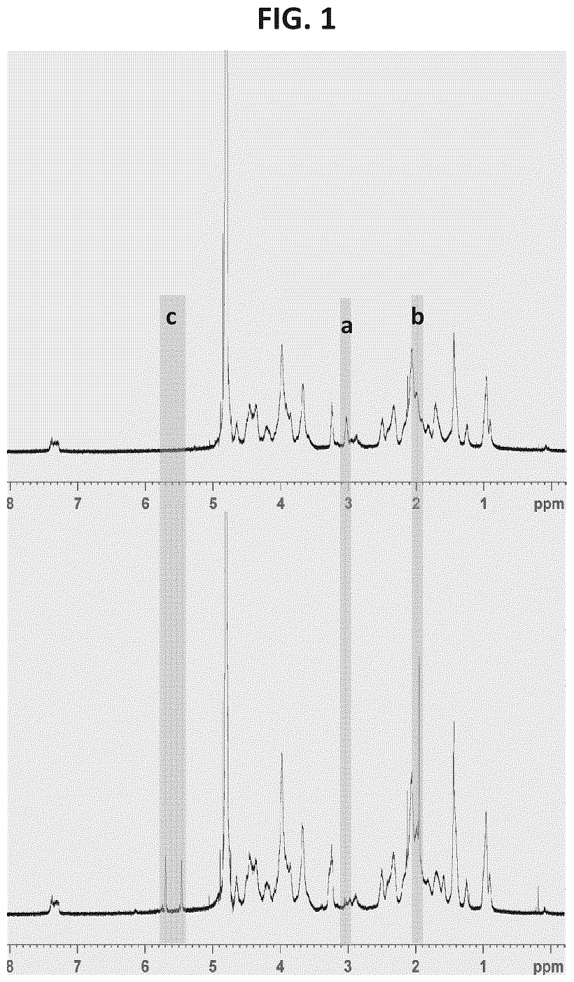

While reducing the present invention to practice, the present inventors have successfully generated curable groups (e.g., (meth)acrylic groups) as substituents of amino acid residues (e.g., lysine residues) of the rhCollagen, and have shown that more than 80% of the lysine residues were modified to feature a methacrylamide group instead of amine group in the side chain thereof, as is exemplified in FIG. 1 and in Table 1.

The rhCollagen that features one or more, preferably a plurality of, curable groups such as (meth)acrylic groups as described herein is also referred to herein throughout, interchangeably, as “curable rhCollagen” or simply as “curable collagen”.

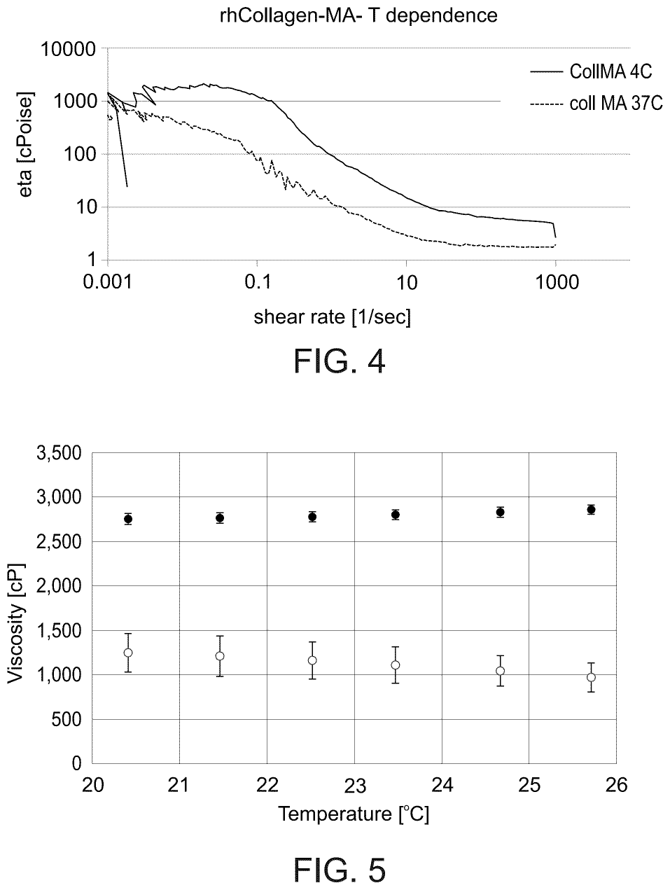

An aqueous solution of the curable rhCollagen was found to exhibit the same relatively low viscosity at low shear rates, and a similar shear-thinning behavior, as exemplified in FIG. 4. Moreover, the curable rhCollagen solution was shown to exhibit a thermal-thinning behavior within a temperature range of working temperatures usable in additive manufacturing processes, and suitable viscosities at this temperature range, as opposed to a thermal-thickening behavior and high viscosities exhibited by a curable tissue-extracted collagen solution, as can be seen in FIG. 5.

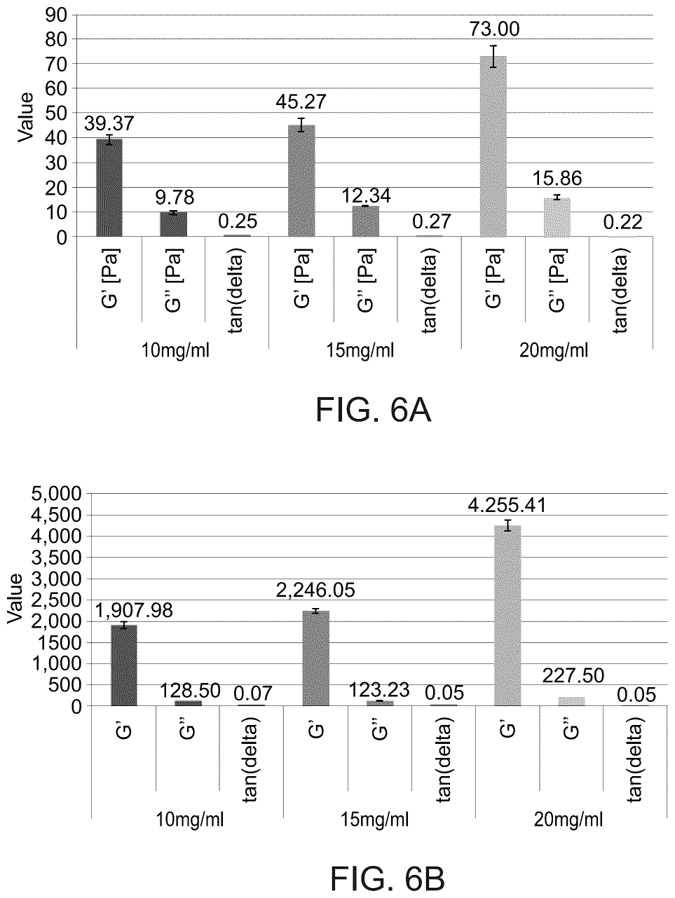

The curable rhCollagen solution was further shown to exhibit good curability and curing stability, providing a cured material with desired mechanical properties, as shown in FIGS. 6A-B, 7 and 8.

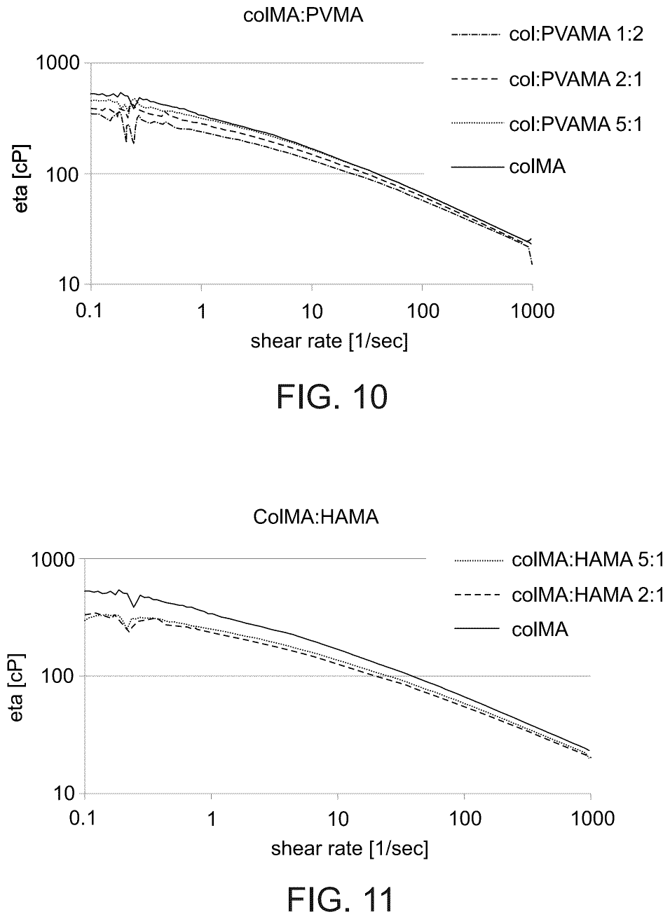

The advantageous properties of the curable rhCollagen were shown to be maintained also in the presence of variable additives, and when used in different solutions (aqueous carriers), as shown in FIGS. 10-16.

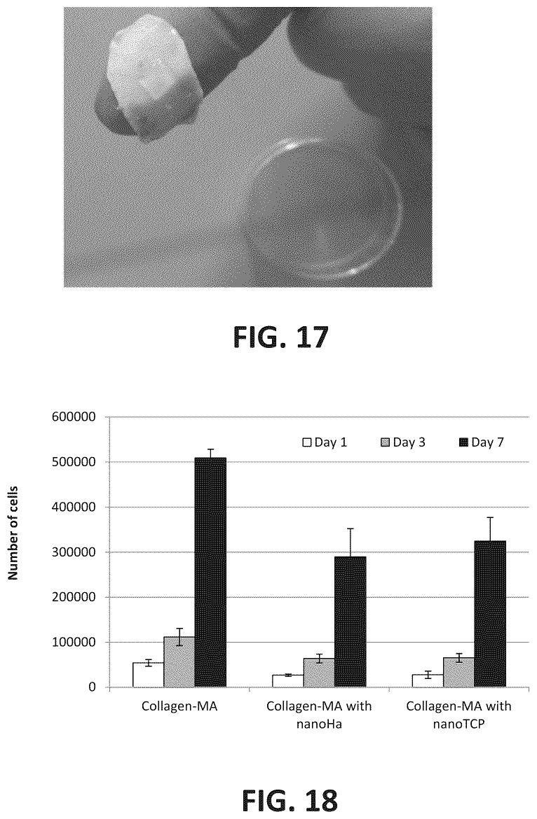

The curable rhCollagen was shown to perform successfully in an in vitro model of transdermal application, as shown in FIG. 17, and as biocompatible, as shown in FIG. 18.

Embodiments of the present invention therefore relate to a composition that comprises a curable recombinant human collagen, to a formulation that comprises a curable recombinant human collagen, and to additive manufacturing processes utilizing same for manufacturing three-dimensional (3D) objects featuring a collagen-based material.

### Additive Manufacturing:

According to an aspect of some embodiments of the present invention, there is provided a process (a method) of additive manufacturing (AM) of a three-dimensional object. According to embodiments of this aspect, the method is effected by sequentially forming a plurality of layers in a configured pattern corresponding to the shape of the object, thereby forming the object. According to embodiments of this aspect, formation of each layer is effected by dispensing at least one uncured building material, and exposing the dispensed building material to a curing condition to thereby form a hardened (cured) material.

Herein throughout, the phrase “building material” encompasses the phrases “uncured building material” or “uncured building material formulation” and collectively describes the materials that are dispensed by sequentially forming the layers, as described herein. This phrase encompasses uncured materials which form the final object, namely, one or more uncured modeling material formulation(s), and optionally also uncured materials used to form a support, namely uncured support material formulations. The building material can also include non-curable materials that preferably do not undergo (or are not intended to undergo) any change during the process, for example, biological materials or components (other than a curable collagen as described herein) and/or other agents or additives as described herein.

The building material that is dispensed to sequentially form the layers as described herein is also referred to herein interchangeably as “printing medium” or “bioprinting medium”.

An uncured building material can comprise one or more modeling material formulations, and can be dispensed such that different parts of the object are made upon hardening (e.g., curing) of different modeling formulations, and hence are made of different hardened (e.g., cured) modeling materials or different mixtures of hardened (e.g., cured) modeling materials.

The method of the present embodiments manufactures three-dimensional objects in a layerwise manner by forming a plurality of layers in a configured pattern corresponding to the shape of the object.

Each layer is formed by an additive manufacturing apparatus which scans a two-dimensional surface and patterns it. While scanning, the apparatus visits a plurality of target locations on the two-dimensional layer or surface, and decides, according to a pre-set algorithm, for each target location or a group of target locations, whether or not the target location or group of target locations is to be occupied by a building material, and which type of a building material is to be delivered thereto. The decision is made according to a computer image of the surface.

When the AM is by three-dimensional inkjet printing, an uncured building material, as defined herein, is dispensed from a dispensing head having a set of nozzles to deposit building material in layers on a supporting structure. The AM apparatus thus dispenses building material in target locations which are to be occupied and leaves other target locations void. The apparatus typically includes a plurality of dispensing heads, each of which can be configured to dispense a different building material (for example, different modeling material formulations, each containing a different biological component; or each containing a different curable material; or each containing a different concentration of a curable material, and/or different support material formulations). Thus, different target locations can be occupied by different building materials (e.g., a modeling formulation and/or a support formulation, as defined herein).

The final three-dimensional object is made of the hardened modeling material or a combination of hardened modeling materials or a combination of hardened modeling material/s and support material/s or modification thereof (e.g., following curing). All these operations are well-known to those skilled in the art of additive manufacturing (also known as solid freeform fabrication).

In some exemplary embodiments of the invention an object is manufactured by dispensing a building material that comprises two or more different modeling material formulations, each modeling material formulation from a different dispensing head of the AM apparatus. The modeling material formulations are optionally and preferably deposited in layers during the same pass of the dispensing heads. The modeling material formulations and/or combination of formulations within the layer are selected according to the desired properties of the object.

An exemplary process according to some embodiments of the present invention starts by receiving 3D printing data corresponding to the shape of the object. The data can be received, for example, from a host computer which transmits digital data pertaining to fabrication instructions based on computer object data, e.g., in a form of a Standard Tessellation Language (STL) or a StereoLithography Contour (SLC) format, Virtual Reality Modeling Language (VRML), Additive Manufacturing File (AMF) format, Drawing Exchange Format (DXF), Polygon File Format (PLY), Digital Imaging and Communications in Medicine (DICOM) or any other format suitable for Computer-Aided Design (CAD).

The process continues by dispensing the building material as described herein in layers, on a receiving medium, using one or more dispensing (e.g., printing) heads, according to the printing data.

The dispensing can be in a form of droplets, or a continuous stream, depending on the additive manufacturing methodology employed and the configuration of choice.

The receiving medium can be a tray of a printing system, or a supporting article or medium made of, or coated by, a biocompatible material, such as support media or articles commonly used in bioprinting, or a previously deposited layer.

In some embodiments, the receiving medium comprises a sacrificial hydrogel or other biocompatible material as a mold to embed the printed object, and is thereafter removed by chemical, mechanical or physical (e.g., heating or cooling) means. Such sacrificial hydrogels can be made of, for example, a Pluronic material or of Gelatin.

Once the uncured building material is dispensed on the receiving medium according to the 3D data, the method optionally and preferably continues by hardening the dispensed formulation(s). In some embodiments, the process continues by exposing the deposited layers to a curing condition. Preferably, the curing condition is applied to each individual layer following the deposition of the layer and prior to the deposition of the previous layer.

As used herein, the term “curing” describes a process in which a formulation is hardened. The hardening of a formulation typically involves an increase in a viscosity of the formulation and/or an increase in a storage modulus of the formulation (G′). In some embodiments, a formulation which is dispensed as a liquid becomes solid or semi-solid (e.g., gel) when hardened. A formulation which is dispensed as a semi-solid (e.g., soft gel) becomes solid or a harder or stronger semi-solid (e.g., strong gel) when hardened.

The term “curing” as used herein encompasses, for example, polymerization of monomeric and/or oligomeric materials and/or cross-linking of polymeric chains (either of a polymer present before curing or of a polymeric material formed in a polymerization of the monomers or oligomers). The product of a curing reaction is therefore typically a polymeric material and/or a cross-linked material. This term, as used herein, encompasses also partial curing, for example, curing of at least 20% or at least 30% or at least 40% or at least 50% or at least 60% or at least 70% of the formulation, as well as 100% of the formulation.

Herein, the phrase “a condition that affects curing” or “a condition for inducing curing”, which is also referred to herein interchangeably as “curing condition” or “curing inducing condition” describes a condition which, when applied to a formulation that contains a curable material, induces a curing as defined herein. Such a condition can include, for example, application of a curing energy, as described hereinafter to the curable material(s), and/or contacting the curable material(s) with chemically reactive components such as catalysts, co-catalysts, and activators.

When a condition that induces curing comprises application of a curing energy, the phrase “exposing to a curing condition” and grammatical diversions thereof means that the dispensed layers are exposed to the curing energy and the exposure is typically performed by applying a curing energy to the dispensed layers.

A “curing energy” typically includes application of radiation or application of heat.

The radiation can be electromagnetic radiation (e.g., ultraviolet or visible light), or electron beam radiation, or ultrasound radiation or microwave radiation, depending on the materials to be cured. The application of radiation (or irradiation) is effected by a suitable radiation source. For example, an ultraviolet or visible or infrared or Xenon or mercury or lamp, or LED source, can be employed, as described herein.

A curable material or system that undergoes curing upon exposure to radiation is referred to herein interchangeably as “photopolymerizable” or “photoactivatable” or “photocurable”.

When the curing energy comprises heat, the curing is also referred to herein and in the art as “thermal curing” and comprises application of thermal energy. Applying thermal energy can be effected, for example, by heating a receiving medium onto which the layers are dispensed or a chamber hosting the receiving medium, as described herein. In some embodiments, the heating is effected using a resistive heater.

In some embodiments, the heating is effected by irradiating the dispensed layers by heat-inducing radiation. Such irradiation can be effected, for example, by means of an IR lamp or Xenon lamp, operated to emit radiation onto the deposited layer.

In some embodiments, heating is effected by infrared radiation applied by a ceramic lamp, for example, a ceramic lamp that produces infrared radiation of from about 3 μm to about 4 μm, e.g., about 3.5 μm.

A curable material or system that undergoes curing upon exposure to heat is referred to herein as “thermally-curable” or “thermally-activatable” or “thermally-polymerizable”.

In some of any of the embodiments described herein, hardening the dispensed formulation(s) comprises exposing the dispensed formulation to a curing condition as described herein in any of the respective embodiments, for example, to irradiation (illumination).

In some embodiments, the exposure to a curing condition is for a short time period, for example, a time period of less than 3 minutes, less than 300 seconds, for example, of from 10 seconds to 240 seconds, or from 10 seconds to 120 seconds, to from 10 seconds to 60 seconds, including an intermediate values and subranges therebetween.

In some of any of the embodiments described herein, the method further comprises exposing the cured modeling material formulation(s) either before or after removal of a support material formulation, if such has been included in the building material, to a post-treatment condition. The post-treatment condition is typically aimed at further hardening the cured modeling material(s). In some embodiments, the post-treatment hardens a partially-cured formulation to thereby obtain a completely cured formulation.

In some embodiments, the post-treatment is effected by exposure to heat or radiation, as described in any of the respective embodiments herein.

Some embodiments contemplate the fabrication of an object by dispensing different formulations from different dispensing heads. These embodiments provide, inter alia, the ability to select formulations from a given number of formulations and define desired combinations of the selected formulations and their properties.

According to the present embodiments, the spatial locations of the deposition of each formulation with the layer are defined, either to effect occupation of different three-dimensional spatial locations by different formulations, or to effect occupation of substantially the same three-dimensional location or adjacent three-dimensional locations by two or more different formulations so as to allow post deposition spatial combination of the formulations within the layer.

The present embodiments thus enable the deposition of a broad range of material combinations, and the fabrication of an object which may consist of multiple different combinations of modeling material formulations, in different parts of the object, according to the properties desired to characterize each part of the object.

A system utilized in additive manufacturing may include a receiving medium and one or more dispensing heads. The receiving medium can be, for example, a fabrication tray that may include a horizontal surface to carry the material dispensed from the printing head. In some embodiments, the receiving medium is made of, or coated by, a biocompatible material, as described herein.

The dispensing head may be, for example, a printing head having a plurality of dispensing nozzles arranged in an array of one or more rows along the longitudinal axis of the dispensing head. The dispensing head may be located such that its longitudinal axis is substantially parallel to the indexing direction.

The additive manufacturing system may further include a controller, such as a microprocessor to control the AM process, for example, the movement of the dispensing head according to a pre-defined scanning plan (e.g., a CAD configuration converted to a Standard Tessellation Language (STL) format and programmed into the controller). The dispensing head may include a plurality of jetting nozzles. The jetting nozzles dispense material onto the receiving medium to create the layers representing cross sections of a 3D object.

In addition to the dispensing head, there may be a source of curing energy, for curing the dispensed building material. The curing energy is typically radiation, for example, UV radiation or heat radiation. Alternatively, there may be means for providing a curing condition other than electromagnetic or heat radiation, for example, means for cooling the dispensed building material of for contacting it with a reagent that promotes curing.

Additionally, the AM system may include a leveling device for leveling and/or establishing the height of each layer after deposition and at least partial solidification, prior to the deposition of a subsequent layer.

According to the present embodiments, the additive manufacturing method described herein is for bioprinting a biological object.

As used herein, “bioprinting” means practicing an additive manufacturing process while utilizing one or more bio-ink formulation(s) that comprise(s) biological components, as described herein, via methodology that is compatible with an automated or semi-automated, computer-aided, additive manufacturing system as described herein (e.g., a bioprinter or a bioprinting system).

Herein throughout, the phrase “modeling material formulation”, which is also referred to herein interchangeably as “modeling formulation” or “modeling material composition” or “modeling composition”, or simply as a “formulation”, or a “composition”, describes a part or all of the uncured building material (printing medium) which is dispensed so as to form the final object, as described herein. The modeling formulation is an uncured modeling formulation, which, upon exposure to a curing condition, forms the object or a part thereof.

In the context of bioprinting, an uncured building material comprises at least one modeling formulation that comprises one or more biological components or materials (e.g., a curable rhCollagen as described herein), and is also referred to herein and in the art as “bio-ink” or “bio-ink formulation”.

In some embodiments, the bioprinting comprises sequential formation of a plurality of layers of the uncured building material in a configured pattern, preferably according to a three-dimensional printing data, as described herein. At least one, and preferably most or all, of the formed layers (before hardening or curing) comprise(s) one or more biological component(s) as described herein (e.g., a curable rhCollagen as described herein). Optionally, at least one of the formed layers (before hardening or curing) comprises one or more non-biological curable materials, and/or non-curable biological or non-biological components, preferably biocompatible materials which do not interfere (e.g., adversely affect) with the biological and/or structural features of the biological components (e.g., collagen) in the printing medium and/or bio-ink.

In some embodiments, the components in the bio-ink or the printing medium, e.g., non-curable and curable materials, and/or the curing condition applied to effect curing, are selected such that they do not significantly affect structural and/or functional properties of the biological components in the bio-ink or printing medium.

In some of any of the embodiments described herein, the building material (e.g., the printing medium) comprises modeling material formulation(s) (bio-ink) and optionally support material formulation(s), and all are selected to include materials or combination of materials that do not interfere with the biological and/or structural features of the biological components.

In some of any of the embodiments described herein, the bioprinting method is configured to effect formation of the layers under conditions that do not significantly affect structural and/or functional properties of the biological components in the bio-ink.

In some embodiments, a bioprinting system for effecting a bioprinting process/method as described herein is configured so as to allow formation of the layers under conditions that do not significantly affect structural and/or functional properties of the biological components in the bio-ink.

In some of any of the embodiments described herein, the additive manufacturing (e.g., bioprinting) process and system are configured such that the process parameters (e.g., temperature, shear forces, shear strain rate) do not interfere with (do not substantially affect) the functional and/or structural features of the biological components.

In some of any of the embodiments described herein, the additive manufacturing process (the bioprinting) is performed at a temperature of at least 10° C., or of at least 20° C., for example, at a temperature that ranges from about 10 to about 40° C., preferably from about 10° C. to 37° C., or from about 20° C. to 37° C., or from about 20° C. to about 30° C., or from about 20° C. to about 28° C., or from about 20° C. to about 25° C., including any intermediate values and subranges therebetween, or at room temperature, or at 37° C.

In some of any of the embodiments described herein, the above-indicated temperatures/temperature ranges are the temperatures at which the building material (e.g., at least a modeling material formulation that comprises a biological component as described herein) are dispensed, that is, a temperature of a dispensing head in the AM system and/or a temperature at which the modeling material formulation is maintained prior to passing in the dispensing head.

In some of any of the embodiments described herein, the AM process is performed without cooling the AM system (e.g., without cooling the dispensing heads and/or a modeling material formulation), to a temperature below room temperature, e.g., a temperature lower than 20° C. or lower than 10° C., or lower than 5° C. (e.g., 4° C.).

In some of any of the embodiments described herein, the AM system is devoid of means for cooling the system or a part thereof (e.g., means for cooling the dispensing heads and/or the modeling material formulation), to a temperature below room temperature, e.g., a temperature lower than 20° C. or lower than 10° C., or lower than 5° C. (e.g., 4° C.).

In some of any of the embodiments described herein, the additive manufacturing process (bioprinting) is performed while applying a shear force that does adversely affect structural and/or functional properties of biological components (e.g., cells). Applying the shear force can be effected by passing the building material (e.g., at least a modeling material formulation that comprises a biological component as described herein) through the dispensing head, and is to be regarded also as subjecting the building material to shear force.

As discussed herein and demonstrated in the Examples section that follow, embodiments of the present invention allow to perform AM bioprinting processes under conditions that do not affect the functional and/or structural features of biological components included in the bio-ink (e.g., at low shear force and room temperature or a physiological temperature), while maintaining the required fluidity (a viscosity that imparts fluidity, e.g., lower than 10,000 centipoises or lower than 5,000 centipoises, or lower than 2,000 centipoises), and while further maintaining the curability of the dispensed building material. The embodiments of the present invention allow a successful operation of bioprinting using any of the known methodology, without being limited to the process parameters required for each such methodology.

The following describes exemplary AM bioprinting methodologies that are usable in the context of embodiments of the present invention.

A bioprinting method and a corresponding system can be any of the methods and systems known in the art for performing additive manufacturing, and exemplary such systems and methods are described hereinabove. A suitable method and system can be selected upon considering its printing capabilities, which include resolution, deposition speed, scalability, bio-ink compatibility and ease-of-use.

Exemplary suitable bioprinting systems usually contain a dispensing system (either equipped with temperature control module or at ambient temperature), and stage (a receiving medium), and a movement along the x, y and z axes directed by a CAD-CAM software. A curing source (e.g., a light or heat source) which applies a curing energy (e.g., by applying light or heat radiation) or a curing condition to the deposition area (the receiving medium) so as to promote curing of the formed layers and/or a humidifier, can also be included in the system. There are printers that use multiple dispensing heads to facilitate a serial dispensing of several materials.

Generally, bioprinting can be effected using any of the known techniques for additive manufacturing. The following lists some exemplary additive manufacturing techniques, although any other technique is contemplated.

### 3D Inkjet Printing:

3D Inkjet printing is a common type of 3D printer for both non-biological and biological (bioprinting) applications. Inkjet printers use thermal or acoustic forces to eject drops of liquid onto a substrate, which can support or form part of the final construct. In this technique, controlled volumes of liquid are delivered to predefined locations, and a high-resolution printing with precise control of (1) ink drops position, and (2) ink volume, which is beneficial in cases of microstructure-printing or when small amounts of bioreactive agents or drugs are added, is received. Inkjet printers can be used with several types of ink, for example, comprising multiple types of biological components and/or bioactive agents. Furthermore, the printing is fast and can be applied onto culture plates.

A bioprinting method that utilizes a 3D inkjet printing system can be operated using one or more bio-ink modeling material formulations as described herein, and dispensing droplets of the formulation(s) in layers, on the receiving medium, using one or more inkjet printing head(s), according to the 3D printing data.

### Extrusion Printing:

This technique uses continuous beads of material rather than liquid droplets. These beads of material are deposited in 2D, the stage (receiving medium) or extrusion head moves along the z axis, and the deposited layer serves as the basis for the next layer. The most common methods for biological materials extrusion for 3D bioprinting applications are pneumatic or mechanical dispensing systems.

### Stereolithography (SLA) and Digital Light Processing (DLP):

SLA and DLP are additive manufacturing technologies in which an uncured building material in a bath is converted into hardened material(s), layer by layer, by selective curing using a light source while the uncured material is later separated/washed from the hardened material. SLA is widely used to create models, prototypes, patterns, and production parts for a range of industries including for Bioprinting.

### Laser-Assisted Printing:

Laser-assisted printing technique, in the version adopted for 3D bioprinting, and is based on the principle of laser-induced forward transfer (LIFT), which was developed to transfer metals and is now successfully applied to biological material. The device consists of a laser beam, a focusing system, an energy absorbing/converting layer and a biological material layer (e.g., cells and/or hydrogel) and a receiving substrate. A laser assisted printer operates by shooting a laser beam onto the absorbing layer which convert the energy into a mechanical force which drives tiny drops from the biological layer onto the substrate. A light source is then utilized to cure the material on the substrate.

Laser assisted printing is compatible with a series of viscosities and can print mammalian cells without affecting cell viability or cell function. Cells can be deposited at a density of up to 10⁸ cells/ml with microscale resolution of a single cell per drop.

### Electrospinning:

Electrospinning is a fiber production technique, which uses electric force to draw charged threads of polymer solutions, or polymer melts.

### Recombinant Human Collagen:

The term “collagen” as used herein, refers to a polypeptide having a triple helix structure and containing a repeating Gly-X—Y triplet, where X and Y can be any amino acid but are frequently the imino acids proline and hydroxyproline. According to one embodiment, the collagen is a type I, II, III, V, XI, or biologically active fragments therefrom.

A collagen of the present invention also refers to homologs (e.g., polypeptides which are at least 50%, at least 55%, at least 60%, at least 65%, at least 70%, at least 75%, at least 80%, at least 85%, at least 87%, at least 89%, at least 91%, at least 93%, at least 95% or more say 100% homologous to collagen sequences listed in Table A as determined using BlastP software of the National Center of Biotechnology Information (NCBI) using default parameters). The homolog may also refer to a deletion, insertion, or substitution variant, including an amino acid substitution, thereof and biologically active polypeptide fragments thereof.

According to a particular embodiment, the collagen is a human collagen.

In another embodiment, the collagen comprises a naturally occurring amino acid sequence of human collagen.

Table A below lists examples of collagen NCBI sequence numbers.

TABLE A

| Exemplary procollagen | |
|---|---|
| NCBI sequence number | SEQ ID NO: |
| P02452 | 1 |
| P08123 | 2 |

The annotation of SEQ ID NO: 1 is as follows:
Amino acids 1-22—signal peptide;
Amino acids 23-161—N-terminal peptide;
Amino acids 162-1218—collagen alpha-1(I) chain;
Amino acids 1219-1464—C-terminal peptide;

The annotation of SEQ ID NO: 2 is as follows:
Amino acids 1-22—signal peptide;
Amino acids 23-79—N-terminal peptide;
Amino acids 80-1119—collagen alpha-2(I) chain;
Amino acids 1120-1366—C-terminal peptide.

According to one embodiment, the collagen of the present invention comprises a sufficient portion of its telopeptides such that under suitable conditions it is capable of forming fibrils.

Thus, for example, the collagen may be atelocollagen, a telocollagen or procollagen.

As used herein, the term “atelocollagen” refers to collagen molecules lacking both the N- and C-terminal propeptides typically comprised in procollagen and at least a portion of its telopeptides, but including a sufficient portion of its telopeptides such that under suitable conditions it is capable of forming fibrils.

The term “procollagen” as used herein, refers to a collagen molecule (e.g. human) that comprises either an N-terminal propeptide, a C-terminal propeptide or both. Exemplary human procollagen amino acid sequences are set forth by SEQ ID NOs: 3, 4, 5 and 6.

The term “telocollagen” as used herein, refers to collagen molecules that lack both the N- and C-terminal propeptides typically comprised in procollagen but still contain the telopeptides. The telopeptides of fibrillar collagen are the remnants of the N- and C-terminal propeptides following digestion with native N/C proteinases.

According to another embodiment, the collagen is devoid of its telopeptides and is not capable of undergoing fibrillogenesis.

According to another embodiment, the collagen is a mixture of the types of collagen above.

According to a particular embodiment, the collagen is genetically engineered using recombinant DNA technology (e.g. human collagen).

Methods of isolating collagen from animals are known in the art. Dispersal and solubilization of native animal collagen can be achieved using various proteolytic enzymes (such as porcine mucosal pepsin, bromelain, chymopapain, chymotrypsin, collagenase, ficin, papain, peptidase, proteinase A, proteinase K, trypsin, microbial proteases, and, similar enzymes or combinations of such enzymes) which disrupt the intermolecular bonds and remove the immunogenic non-helical telopeptides without affecting the basic, rigid triple-helical structure which imparts the desired characteristics of collagen (see U.S. Pat. Nos. 3,934,852; 3,121,049; 3,131,130; 3,314,861; 3,530,037; 3,949,073; 4,233,360 and 4,488,911 for general methods for preparing purified soluble collagen). The resulting soluble collagen can be subsequently purified by repeated precipitation at low pH and high ionic strength, followed by washing and re-solublization at low pH.

Plants expressing collagen chains and procollagen are known in the art, see for example, WO06035442A3; Merle et al., FEBS Lett. 2002-3-27; 515(1-3):114-8. PMID: 11943205; and Ruggiero et al., 2000, FEBS Lett. 2000-3-3; 469(1):132-6. PMID: 10708770; and U.S. Patent Applications Publication Nos. 2002/098578 and 2002/0142391, as well as U.S. Pat. No. 6,617,431 each of which are incorporated herein by reference.

It will be appreciated that the present invention also contemplates genetically modified forms of collagen/atelocollagen—for example collagenase-resistant collagens and the like [Wu et al., Proc Natl. Acad Sci, Vol. 87, p. 5888-5892, 1990].

Recombinant procollagen or telocollagen (e.g. human) may be expressed in any non-animal cell, including but not limited to plant cells and other eukaryotic cells such as yeast and fungus.

Plants in which procollagen or telocollagen may be produced (i.e. expressed) may be of lower (e.g. moss and algae) or higher (vascular) plant species, including tissues or isolated cells and extracts thereof (e.g. cell suspensions). Preferred plants are those which are capable of accumulating large amounts of collagen chains, collagen and/or the processing enzymes described herein below. Such plants may also be selected according to their resistance to stress conditions and the ease at which expressed components or assembled collagen can be extracted. Examples of plants in which human procollagen may be expressed include, but are not limited to tobacco, maize, alfalfa, rice, potato, soybean, tomato, wheat, barley, canola, carrot, lettuce and cotton.

Production of recombinant procollagen is typically effected by stable or transient transformation with an exogenous polynucleotide sequence encoding human procollagen.

Exemplary polynucleotide sequences encoding human procollagen are set forth by SEQ ID NOs: 7, 8, 9 and 10.

Production of human telocollagen is typically effected by stable or transient transformation with an exogenous polynucleotide sequence encoding human procollagen and at least one exogenous polynucleotide sequence encoding the relevant protease. Alternatively, a protease may be added following isolation of the recombinant procollagen.

The stability of the triple-helical structure of collagen requires the hydroxylation of prolines by the enzyme prolyl-4-hydroxylase (P4H) to form residues of hydroxyproline within the collagen chain. Although plants are capable of synthesizing hydroxyproline-containing proteins, the prolyl hydroxylase that is responsible for synthesis of hydroxyproline in plant cells exhibits relatively loose substrate sequence specificity as compared with mammalian P4H. Thus, production of collagen containing hydroxyproline only in the Y position of Gly-X—Y triplets requires co-expression of collagen and human or mammalian P4H genes [Olsen et al, Adv Drug Deliv Rev. 2003-11-28; 55(12):1547-67].

Thus, according to one embodiment, the procollagen or telocollagen is expressed in a subcellular compartment of a plant that is devoid of endogenous P4H activity.

As used herein, the phrase “subcellular compartment devoid of endogenous P4H activity” refers to any compartmentalized region of the cell which does not include plant P4H or an enzyme having plant-like P4H activity. According to one embodiment, the subcellular compartment is a vacuole, an apoplast or a chloroplast. According to a particular embodiment, the subcellular compartment is a vacuole.

According to another embodiment, the subcellular compartment is an apoplast.

Accumulation of the expressed procollagen in a subcellular compartment devoid of endogenous P4H activity can be effected via any one of several approaches.

For example, the expressed procollagen/telocollagen can include a signal sequence for targeting the expressed protein to a subcellular compartment such as the apoplast or an organelle (e.g. chloroplast).

Examples of suitable signal sequences include the chloroplast transit peptide (included in Swiss-Prot entry P07689, amino acids 1-57) and the Mitochondrion transit peptide (included in Swiss-Prot entry P46643, amino acids 1-28). Targeting to the vacuole may be achieved by fusing the polynucleotide sequence encoding the collagen to a vacuolar targeting sequence—for example using the vacuolar targeting sequence of the thiol protease aleurain precursor (NCBI accession P05167 GI:113603): —MAHARVLLLALAVLATAAVAVASSSSFADSNPIRPVTDRAASTLA (SEQ ID NO: 14). Typically, the polynucleotide sequence encoding the collagen also comprises an ER targeting sequence. In one embodiment, the ER targeting sequence is native to the collagen sequence. In another embodiment, the native ER targeting sequence is removed and a non-native ER targeting sequence is added. The non-native ER targeting sequence may be comprised in the vacuolar targeting sequence. It will be appreciated, for it to traverse the ER and move on to the vacuole, the collagen sequence should be devoid of an ER retention sequence.

Alternatively, the sequence of the procollagen can be modified in a way which alters the cellular localization of the procollagen when expressed in plants.

The present invention contemplates genetically modified cells co-expressing both human procollagen and a P4H. In one embodiment, the P4H is capable of correctly hydroxylating the procollagen alpha chain(s) [i.e. hydroxylating only the proline (Y) position of the Gly-X—Y triplets]. P4H is an enzyme composed of two subunits, alpha and beta as set forth in Genbank Nos. P07237 and P13674. Both subunits are necessary to form an active enzyme, while the beta subunit also possesses a chaperon function.

The P4H expressed by the genetically modified cells of the present invention is preferably a human P4H. An exemplary polynucleotide sequence which encodes human P4H is SEQ ID Nos: 11 and 12. In addition, P4H mutants which exhibit enhanced substrate specificity, or P4H homologues can also be used. A suitable P4H homologue is exemplified by an Arabidopsis oxidoreductase identified by NCBI accession no: NP_179363.

Since it is essential that P4H co-accumulates with the expressed procollagen chain, the coding sequence thereof is preferably modified accordingly (e.g. by addition or deletion of signal sequences). Thus, the present invention contemplates using P4H polynucleotide sequences that are fused to vacuole targeting sequences. It will be appreciated that for targeting to the vacuole, when an endogenous ER retention sequence is present, it should be removed prior to expression.

In mammalian cells, collagen is also modified by Lysyl hydroxylase, galactosyltransferase and glucosyltransferase. These enzymes sequentially modify lysyl residues in specific positions to hydroxylysyl, galactosylhydroxylysyl and glucosylgalactosyl hydroxylysyl residues at specific positions. A single human enzyme, Lysyl hydroxylase 3 (LH3), as set forth in Genbank No. 060568, can catalyze all three consecutive modifying steps as seen in hydroxylysine-linked carbohydrate formation.

Thus, the genetically modified cells of the present invention may also express mammalian LH3 (optionally fused to vacuole targeting sequences). It will be appreciated that for targeting to the vacuole, the endogenous ER retention sequence is removed prior to expression.

An LH3 encoding sequence such as that set forth by SEQ ID NO: 13, can be used for such purposes.

The procollagen(s) and modifying enzymes described above can be expressed from a stably integrated or a transiently expressed nucleic acid construct which includes polynucleotide sequences encoding the procollagen alpha chains and/or modifying enzymes (e.g. P4H and LH3) positioned under the transcriptional control of functional promoters. Such a nucleic acid construct (which is also termed herein as an expression construct) can be configured for expression throughout the whole organism (e.g. plant, defined tissues or defined cells), and/or at defined developmental stages of the organism. Such a construct may also include selection markers (e.g. antibiotic resistance), enhancer elements and an origin of replication for bacterial replication.

There are various methods for introducing nucleic acid constructs into both monocotyledonous and dicotyledenous plants (Potrykus, I., Annu. Rev. Plant. Physiol., Plant. Mol. Biol. (1991) 42:205-225; Shimamoto et al., Nature (1989) 338:274-276). Such methods rely on either stable integration of the nucleic acid construct or a portion thereof into the genome of the plant, or on transient expression of the nucleic acid construct, in which case these sequences are not inherited by the plant's progeny.

In addition, several methods exist in which a nucleic acid construct can be directly introduced into the DNA of a DNA-containing organelle such as a chloroplast.

There are two principle methods of effecting stable genomic integration of exogenous sequences, such as those included within the nucleic acid constructs of the present invention, into plant genomes:

(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.

(ii) Direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

There are various methods of direct DNA transfer into plant cells. In electroporation, protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals, tungsten particles or gold particles, and the microprojectiles are physically accelerated into cells or plant tissues.

Regardless of the transformation technique employed, once collagen-expressing progeny are identified, such plants are further cultivated under conditions which maximize expression thereof. Progeny resulting from transformed plants can be selected, by verifying presence of exogenous mRNA and/or polypeptides by using nucleic acid or protein probes (e.g. antibodies). The latter approach enables localization of the expressed polypeptide components (by for example, probing fractionated plants extracts) and thus also verifies the plant's potential for correct processing and assembly of the foreign protein.

Following cultivation of such plants, the telopeptide-comprising collagen is typically harvested. Plant tissues/cells are preferably harvested at maturity, and the procollagen molecules are isolated using extraction approaches. Preferably, the harvesting is effected such that the procollagen remains in a state that it can be cleaved by protease enzymes. According to one embodiment, a crude extract is generated from the transgenic plants of the present invention and subsequently contacted with the protease enzymes.

As mentioned, the propeptide or telopeptide-comprising collagen may be incubated with a protease to generate atelocollagen or collagen prior to solubilization. It will be appreciated that the propeptide or telopeptide-comprising collagen may be purified from the genetically engineered cells prior to incubation with protease, or alternatively may be purified following incubation with the protease. Still alternatively, the propeptide or telopeptide-comprising collagen may be partially purified prior to protease treatment and then fully purified following protease treatment. Yet alternatively, the propeptide or telopeptide-comprising collagen may be treated with protease concomitant with other extraction/purification procedures.

Exemplary methods of purifying or semi-purifying the telopeptide-comprising collagen of the present invention include, but are not limited to salting out with ammonium sulfate or the like and/or removal of small molecules by ultrafiltration.

According to one embodiment, the protease used for cleaving the recombinant propeptide or telopeptide comprising collagen is not derived from an animal. Exemplary proteases include, but are not limited to certain plant derived proteases e.g. ficin (EC 3.4.22.3) and certain bacterial derived proteases e.g. subtilisin (EC 3.4.21.62), neutrase. The present inventors also contemplate the use of recombinant enzymes such as rhTrypsin and rhPepsin. Several such enzymes are commercially available e.g. Ficin from Fig tree latex (Sigma, catalog #F4125 and Europe Biochem), Subtilisin from Bacillus licheniformis (Sigma, catalog #P5459) Neutrase from bacterium Bacillus amyloliquefaciens (Novozymes, catalog #PW201041) and TrypZean™, a recombinant human trypsin expressed in corn (Sigma catalog #T3449).

In some of any of the embodiments described herein, the recombinant human collagen is a recombinant human type I collagen.

In some of any of the embodiments described herein, the recombinant human collagen is a plant-derived recombinant human collagen and in some embodiments the plant is tobacco.

In some of any of the embodiments described herein, the recombinant human collagen is a recombinant human type I collagen comprising two α1 units having the amino acid sequence which is at least 90% homologous, at least 91% homologous, 92% homologous, at least 93% homologous, at least 94% homologous, at least 95% homologous, at least 96% homologous, at least 97% homologous, at least 98% homologous, at least 99% homologous or 100% homologous to the sequence as set forth in SEQ ID NO:15 as determined using BlastP software of the National Center of Biotechnology Information (NCBI) using default parameters), and one α2 unit having the amino acid sequence which is at least 90% homologous, at least 91% homologous, 92% homologous, at least 93% homologous, at least 94% homologous, at least 95% homologous, at least 96% homologous, at least 97% homologous, at least 98% homologous, at least 99% homologous or 100% homologous to the sequence as set forth in SEQ ID NO:6. According to a particular embodiment, the type I collagen consists of two α1 units which consists of the sequence as set forth in SEQ ID NO: 15 and one α2 unit consisting of the sequence as set forth in SEQ ID NO:6, as determined using BlastP software of the National Center of Biotechnology Information (NCBI) using default parameters).

In some of any of the embodiments described herein, the α1 unit is encoded by a polynucleotide sequence being at least which is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 93%, at least about 94%, at least about 95%, at least about 96%, at least about 97%, at least about 98%, at least about 99%, e.g., 100% identical to the nucleic acid sequence as set forth in SEQ ID NO: 16. The α2 unit is encoded by a polynucleotide sequence being at least which is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 93%, at least about 94%, at least about 95%, at least about 96%, at least about 97%, at least about 98%, at least about 99%, e.g., 100% identical to the nucleic acid sequence as set forth in SEQ ID NO: 10.

Identity (e.g., percent homology) can be determined using any homology comparison software, including for example, the BlastN software of the National Center of Biotechnology Information (NCBI) such as by using default parameters.

In some of any of the embodiments described herein, the human recombinant collagen (rhCollagen) as described herein in any of the respective embodiments is a monomeric rhCollagen.

By “monomeric” it is meant a rhCollagen as described herein which is soluble in an aqueous solution and does not form fibrillar aggregates.

In some of any of the embodiments described herein, the human recombinant collagen (rhCollagen) as described herein in any of the respective embodiments is a fibrillar rhCollagen.

By “fibrillar” it is meant a rhCollagen as described herein which is in a form of fibrillar aggregates in an aqueous solution containing same. Typically, but not obligatory, fibrillar rhCollagen is formed by subjecting monomeric rhCollagen to a fibrillogenesis buffer, typically featuring a basic pH. An exemplary procedure for forming fibrillar rhCollagen, is described in the Examples section that follows.

### Curable Collagen:

According to an aspect of some embodiments of the present invention there is provided a composition that comprises a curable collagen. According to some embodiments, the composition is usable in additive manufacturing of a 3D object as described herein (e.g., in bioprinting). According to some embodiments, the composition is usable, or is for use, in the preparation of a modeling material formulation for an additive manufacturing process (e.g., bioprinting).

By “curable” it is meant herein a material that is capable of undergoing curing, or hardening, as defined herein, when exposed to a suitable curing condition.

A curable material is typically hardened or cured by undergoing polymerization and/or cross-linking.

Curable materials are typically polymerizable materials, which undergo polymerization and/or cross-linking when exposed to a suitable curing condition or a suitable curing energy (a suitable energy source). Alternatively, curable materials are thermo-responsive materials, which solidify or harden upon exposure to a temperature change (e.g., heating or cooling). Optionally, curable materials are made of small particles (e.g., nanoparticles or nanoclays) which can undergo curing to form a hardened material. Further optionally, curable materials are biological materials which undergo a reaction to form a hardened or solid material upon a biological reaction (e.g., an enzymatically-catalyzed reaction).

In some of any of the embodiments described herein, a curable material is a photopolymerizable material, which polymerizes and/or undergoes cross-linking upon exposure to radiation, as described herein, and in some embodiments the curable material is a UV-curable material, which polymerizes or undergoes cross-linking upon exposure to UV-vis radiation, as described herein.

In some of any of the embodiments described herein, when a curable material is exposed to a curing condition (e.g., radiation, reagent), it polymerizes by any one, or combination, of chain elongation, entanglement and cross-linking. The cross-linking can be chemical and/or physical.

In some of any of the embodiments described herein, a curable material can be a mono-functional curable material or a multi-functional curable material.

Herein, a mono-functional curable material comprises one curable group—a functional group that can undergo polymerization, entanglement and/or cross-linking when exposed to a curing condition (e.g., radiation, presence of calcium ions).

A multi-functional curable material comprises two or more, e.g., 2, 3, 4 or more, curable groups. Multi-functional curable materials can be, for example, di-functional, tri-functional or tetra-functional curable materials, which comprise 2, 3 or 4 curable groups, respectively.

By “curable collagen” it is meant a collagen as described herein in any of the respective embodiments (human recombinant collagen), which features one or more curable groups as defined herein. According to some of any of the embodiments described herein, the curable collagen is a multi-functional curable material that comprises a plurality of curable groups, as defined herein.

The terms “curable collagen”, “curable rhCollagen” and “rhCollagen featuring one or more (or at least one) curable groups” are used herein interchangeably.

According to some of any of the embodiments described herein, the curable collagen comprises an amino acid sequence as described herein in any of the respective embodiments, and features one or more, preferably a plurality of, curable groups generated at least a portion of the amino acid residues forming the collagen, preferably by covalent attachment of a compound that comprises a curable group to functional groups of the side chains of the amino acid residues. Alternatively, or in addition, curable groups can be generated at the N-terminus and/or C-terminus of one or more the units forming the collagen, for example, by covalent attachment of compound that comprises a curable group to a respective amine or carboxylate.

According to some of any of the embodiments described herein, at least a portion of the curable groups in a curable collagen as described herein are cross-linkable groups, which undergo cross-linking when exposed to a curing condition.

In some embodiments, curable groups can undergo polymerization and/or cross-linking via free-radical mechanism.

Exemplary such curable groups include acrylic groups, including acrylate, methacrylate, acrylamide and methacrylamide groups. Other free-radical curable groups may include thiols, vinyl ethers and other groups that feature a reactive double bond.

In some embodiments, curable groups can undergo polymerization and/or cross-linking via other mechanisms, such as cationic polymerization, or (cationic or anionic) ring opening polymerization. Exemplary such curable groups include, but are not limited to, epoxy-containing groups, caprolactam, caprolactone, oxetane, and vinyl ether.

Other curable groups can include, for example, formation of amide bonds between functional carboxylate and amine group (each being a curable group that reacts with the other and can effect cross-linking); formation of urethane between isocyanate groups and hydroxyl groups via polycondensation in the presence of a catalyst and/or upon exposure to UV radiation; and formation of disulfide bonds between two thiols.

Any other curable groups are contemplated.

The curable groups in the curable collagen can be generated by means of chemical reactions between a material that comprises or can generate the curable group(s) when reacted with chemically-compatible functional groups present in the collagen, as described herein, either directly, or be means of a spacer or a linker, using chemistries well known in the art. For example, a material that comprises a curable group and a functional group can be reacted with a compatible functional group in the collagen, for example, a functional group in an amino acid side chain, such that the curable group is a substituent of the amino acid side chain.

In some embodiments, a compatible functional group is first generated within the collagen by chemical modification of chemical groups of the collagen, and is that reacted with a material that comprises or generates a curable group upon the reaction.

Whenever a curable collagen comprises more than one curable group, the curable groups can be the same of different.

According to some of any of the embodiments described herein, at least a portion, or all, of the curable groups in a curable collagen of the present embodiments are photopolymerizable groups (e.g., UV-curable groups) that are capable of undergoing polymerization and/or cross-linking upon exposure to irradiation as described herein.

According to some of any of the embodiments described herein there the curable group is a photocurable or photopolymerizable group (e.g., an acrylate or methacrylate).

Alternatively, or in addition, the curable group is a thiol-containing group, which provides disulfide bridge upon curing.

Alternatively, or in addition, the curable group or moiety is cured upon undergoing a chemical reaction, such as glycation of conjugation (using coupling agents such as EDC).

According to some embodiments, the curable groups comprise an amine and a carboxyl group which form peptide bonds upon curing.

According to some of any of the embodiments described herein, at least a portion, or all, of the curable groups in a curable collagen of the present embodiments are acrylic groups, as defined herein.

According to some of any of the embodiments described herein, an acrylic group such as metharylamide can be generated by reacting an acrylate or methacrylate (e.g., acrylic acid, methacrylic acid, acrylic or methacrylic ester, acrylic or methacrylic anhydride) with an amine functional group (of, for example, lysine residues).

According to some of any of the embodiments of the present invention, the number of the curable groups in a curable collagen as described herein can determine the degree of curing (e.g., the degree of cross-linking) and can be manipulated in order to achieve a desired curing (e.g., cross-linking) degree.

According to some of any of the embodiments described herein, the curable collagen features a plurality of acrylamide or methacrylamide curable groups generated by reacting with lysine residues as described herein.

According to some of any of the embodiments described herein, the curable collagen features a plurality of acrylamide or methacrylamide curable groups substituting the amine groups of lysine residues in the collagen.

In some embodiments, at least 50%, or at least 60%, or at least 70%, of the lysine residues in the collagen are substituted by a methacrylamide or acrylamide group. In some embodiments, the curable collagen features from 70% to 100%, or from 80% to 100%, or from 90% to 100%, of its lysine residues substituted by a methacrylamide or acrylamide group, including any intermediate values and subranges therebetween.

According to an aspect of some embodiments of the present invention there is provided a process of preparing a curable rhCollagen as described herein. The process is effected, in some embodiments, by reacting a material that comprises a curable group or which generates a curable group with the rhCollagen, in accordance with the embodiments described herein.

The number of curable groups in the rhCollagen can be controlled by manipulating the amount of the material reacted with the rhCollagen for generating the curable groups.

In some embodiments, the material is used in a molar excess with respect to the respective functional groups that are reacted therewith, for example, in a mol ratio of 2:1, 5:1, 10:1, 15:1, 20:1 30:1, 50:1, or, for example, from 1.1:1 to 50:1, including any intermediate values and subranges therebetween, with respect to the chemically compatible functional groups in the rhCollagen (e.g., the amino acid residues featuring such functional groups), or with respect to the rhCollagen.

According to some of any of the embodiments described herein, the curable collagen is a recombinant human type I collagen as described herein in any of the respective embodiments and any combination thereof.

### Modeling Material Formulation(s):

According to the present embodiments described herein, in at least one of the layers formed during the AM (bioprinting process), the dispensed modeling material formulation comprises a biological component or material as described herein and is regarded as bio-ink, as described herein.

According to the present embodiments, the building material comprises at least one modeling material formulation that comprises a curable recombinant human collagen, as described herein in any of the respective embodiments. Such a modeling material formulation is also referred to herein as rhCollagen-containing formulation.

According to some of any of the embodiments described herein, the rhCollagen-containing formulation further comprises a carrier, and in some of these embodiments, the carrier is an aqueous carrier.

The aqueous carrier can be water, a buffer featuring pH in a range of from about 4 to about 10, or from about 6 to about 8, or from about 7 to about 7.4, a basic aqueous solution or an acidic aqueous solution.

The aqueous carrier can comprise salts and other water-soluble materials at varying concentrations. In some embodiments, a concentration of a salt in the carrier ranges from about 0.1 mM to about 0.2 M, or from about 0.1 mM to about 0.1 M, or from about 0.1 mM to about 100 mM, or from about 0.1 mM to about 50 mM, or from about 0.1 mM to about 20 mM, including an intermediate values and subranges therebetween.

In some embodiments, the aqueous carrier comprises salts at physiologically acceptable concentrations, such that the formulation features osmolarity around a physiological osmolarity.

In some embodiments the aqueous carrier comprises a phosphate salt, for example, a sodium phosphate monobasic (NaH₂PO₄) and/or a sodium phosphate dibasic (sodium hydrogen phosphate; Na₂HPO₄). In some embodiments, the total concentration of the phosphate salt(s) is about 0.1 M.

In some embodiments, the aqueous carrier comprises NaCl or any other physiologically acceptable salt.

In some embodiments, the aqueous carrier comprises a phosphate buffer and in some embodiments, the aqueous carrier comprises a phosphate buffer saline, which comprises sodium phosphate monobasic and/or sodium phosphate dibasic and NaCl.

The phosphate buffer saline (PBS) can be a commercially available PBS (e.g., DPBS) or a custom-made buffer featuring a desirable pH and/or osmolarity.

In exemplary embodiments, the aqueous carrier comprises a phosphate buffer that comprises a phosphate sodium salt as described herein at a concentration of about 0.1 M and NaCl at a concentration of from about 0 mM to about 200 mM, including any intermediate value and subranges therebetween.

Any other buffers are also usable in the context of the present embodiments.

In some of any of the embodiments described herein, the aqueous carrier comprises an acid.

In some embodiments, a concentration of the acid is lower than 100 mM, and can be, for example, of from 0.1 mM to 50 mM, or from 0.1 mM to 30 mM, or from 0.1 mM to 40 mM, or from 0.1 mM to 30 mM, or from 1 to 30 mM, or from 10 to 30 Mm, including any intermediate values and subranges therebetween.

The acid can be an inorganic acid (e.g., HCl) or an organic acid, preferably which is water soluble at the above-indicated concentrations (e.g., acetic acid).

In some of any of the embodiments described herein, the aqueous carrier comprises a culturing medium. The culturing medium can be a commercially available culturing medium or a custom-made culturing medium. The culture medium can be any liquid medium which allows at least cell survival. Such a culture medium can include, for example, salts, sugars, amino acids and minerals in the appropriate concentrations and with various additives and those of skills in the art are capable of determining a suitable culture medium to specific cell types. Non-limiting examples of such culture medium include, phosphate buffered saline, DMEM, MEM, RPMI 1640, McCoy's 5A medium, medium 199 and IMDM (available e.g., from Biological Industries, Beth Ha'emek, Israel; Gibco-Invitrogen Corporation products, Grand Island, NY, USA).

The culture medium may be supplemented with various antibiotics (e.g., Penicillin and Streptomycin), growth factors or hormones, specific amino acids (e.g., L-glutamin) cytokines and the like.

In some of any of the embodiments described herein, a concentration of the curable recombinant human collagen in the modeling material formulation containing same ranges from 0.5 mg/mL to 50 mg/mL, or from 0.5 mg/mL to 20 mg/mL, or from 1 mg/mL to 50 mg/mL, or from 1 mg/mL to 50 mg/mL, or from 1 mg/mL to 40 mg/mL, or from 1 mg/mL to 30 mg/mL, or from 2 mg/mL to 20 mg/mL, or from 5 mg/mL to 15 mg/mL, including any intermediate values and subranges therebetween.

A concentration of the curable rhCollagen in a modeling material formulation containing same can affect the rheological properties of the formulation and of the hardened formulation obtained upon dispensing, and can be manipulated in accordance with AM methodology and conditions employed and desired properties of the final object or a portion thereof.

According to some of any of the embodiments described herein, the modeling material formulation features a shear-thinning behavior and is a shear-thinning formulation.

According to some of any of the embodiments described herein, the modeling material formulation features a thermal-thinning behavior and a thermal-thinning formulation.

The term “shear-thinning” describes a property of a fluidic material that is reflected by a decrease in its viscosity (increase in its fluidity) upon application of shear forces (under shear strain). In some of the present embodiments, a shear-thinning material is such that exhibits a significant, e.g., at least 100%, reduction in its shear modulus upon increasing the shear strain from about 1% to above 50%. Shear-thinning materials therefore exhibit a shear-dependent viscosity profile.

The term “thermal-thinning” describes a property of a fluidic material that is reflected by a decrease in its viscosity (increase in its fluidity) upon application of heat energy (increase in temperature). In some of the present embodiments, thermal-thinning materials feature a decrease in viscosity by at least 10% upon a temperature increase of 5° C. Thermal-thinning materials therefore exhibit a temperature-dependent viscosity profile.

According to some of any of the embodiments described herein, the rhColagen-containing formulation is both shear-thinning and thermal-thinning formulation, as defined herein.

According to some of any of the embodiments described herein, the modeling material formulation features a viscosity of no more than 2,000 centipoises, or no more than 1,500 centipoises, at a zero shear rate, at 37° C., when determined using a rheometer as described in the Examples section that follows.

According to some of any of the embodiments described herein, the modeling material formulation features a viscosity of no more than 2000 centipoises, or no more than 1,500 centipoises, at a shear rate of 5 1/sec, at room temperature, when determined using a rheometer as described in the Examples section that follows.

According to some of any of the embodiments described herein, the modeling material formulation features the above-indicated viscosity values when a concentration of the curable collagen is at least 3 mg/mL, or at least 5 mg/mL, for example, is 10 mg/mL.

According to some of any of the embodiments described herein, the modeling material formulation features a viscosity profile at 37° C. which is similar to its viscosity profile at 4° C.

According to some of any of the embodiments described herein, the modeling material formulation features a viscosity at 37° C. which is lower by at least 10%, or at least 20%, than its viscosity at 4° C., when measured at a shear rate of from 1 to 10 [1/sec], for the same concentration of the curable rhCollagen.

According to some of any of the embodiments described herein, the modeling material formulation features, when hardened, storage modulus (G′) of at least 1,000 Pa.

According to some of any of the embodiments described herein, the modeling material features, when hardened (e.g., when exposed a curing condition as described herein in any of the respective embodiments), an increase of at least 10-folds of its storage modulus (G′). According to some of any of the embodiments described herein, the modeling material formulation provides, when hardened, a hydrogel material, formed upon cross-linking of the rhCollagen within the aqueous carrier.

Herein and in the art, the term “hydrogel” describes a three-dimensional fibrous network containing at least 20%, typically at least 50%, or at least 80%, and up to about 99.99% (by mass) water. A hydrogel can be regarded as a material which is mostly water, yet behaves like a solid or semi-solid due to a three-dimensional crosslinked solid-like network, made of polymeric chains (e.g., collagen chains), within the liquid dispersing medium. The polymeric chains are inter-connected (crosslinked) by chemical bonds (covalent, hydrogen and ionic/complex/metallic bonds, typically covalent bonds).

Herein throughout, whenever polymeric chains or a polymeric material is described, it encompasses polymeric biological materials (e.g., macromolecules) such as peptides, proteins, oligonucleotides and nucleic acids.

Hydrogels may take a physical form that ranges from soft, brittle and weak to hard, elastic and tough material. Soft hydrogels may be characterized by rheological parameters including elastic and viscoelastic parameters, while hard hydrogels are suitably characterized by tensile strength parameters, elastic, storage and loss moduli, as these terms are known in the art.

The softness/hardness of a hydrogel is governed inter alia by the chemical composition of the polymer chains, the “degree of cross-linking” (number of interconnected links between the chains), the aqueous media content and composition, and temperature.

A hydrogel, according to some embodiments of the present invention, may contain macromolecular polymeric and/or fibrous elements which are not chemically connected to the main crosslinked network but are rather mechanically intertwined therewith and/or immersed therein. Such macromolecular fibrous elements can be woven (as in, for example, a mesh structure), or non-woven, and can, in some embodiments, serve as reinforcing materials of the hydrogel's fibrous network. Non-limiting examples of such macromolecules include polycaprolactone, gelatin, gelatin methacrylate, alginate, alginate methacrylate, chitosan, chitosan methacrylate, glycol chitosan, glycol chitosan methacrylate, hyaluronic acid (HA), HA methacrylate, and other non-crosslinked natural or synthetic polymeric chains and the likes. Alternatively, or in addition, such macromolecules are chemically connected to the main crosslinked network of the hydrogel, for example, by acting as a cross-linking agent, or by otherwise forming a part of the three-dimensional network of the hydrogel.

In some embodiments, the hydrogel is porous and in some embodiments, at least a portion of the pores in the hydrogel are nanopores, having an average volume at the nanoscale range.

According to some of any of the embodiments described herein, the rhCollagen-containing modeling material formulation further comprises one or more additional materials, including, for example, one or more additional curable materials, one or more non-curable materials and/or one or more biological components or materials.

According to some of any of the embodiments described herein, the printing media (the building material) comprises one or more additional materials, including, for example, one or more additional curable materials, one or more non-curable materials and/or one or more biological components.

According to some of any of the embodiments described herein, the additional materials are included in the rhCollagen-containing formulation or in one or more other modeling material formulations.

Additional curable materials that can be included in the rhCollagen formulation or in one or more other modeling material formulations can be any curable material as defined herein, and is preferably a biocompatible material.

In some embodiments the additional curable material is or comprises a hydrogel, as defined herein, which can form a hardened modeling material, typically upon further cross-linking and/or co-polymerization, when exposed to a curing condition at which the cross-linking and/or co-polymerization reaction occurs. Such curable materials are also referred to herein as hydrogel curable materials.

In some of any of the embodiments described herein, a curable material is or comprises a hydrogel forming material, as defined herein, which can form a hydrogel as a hardened modeling material, typically upon cross-linking, entanglement, polymerization and/or co-polymerization, when exposed to a curing condition at which the cross-linking, polymerization and/or co-polymerization, and/or entanglement reaction occurs. Such curable materials are also referred to herein as hydrogel-forming curable materials or as gel-forming materials.

The hydrogel, according to embodiments of the present invention, can be of biological origin or synthetically prepared.

According to some embodiments of the present invention, the hydrogel is biocompatible, and is such that when a biological moiety is impregnated or accumulated therein, an activity of the biological moiety is maintained, that is, a change in an activity of the biological moiety is no more than 30%, or no more than 20%, or no more than 10%, compared to an activity of the biological moiety in a physiological medium.

Exemplary polymers or co-polymers usable for forming a hydrogel according to the present embodiments include polyacrylates, polymethacrylates, polyacrylamides, polymethacrylamides, polyvinylpyrrolidone and copolymers of any of the foregoing. Other examples include polyethers, polyurethanes, and poly(ethylene glycol), functionalized by cross-linking groups or usable in combination with compatible cross linking agents.

Some specific, non-limiting examples, include: poly(2-vinylpiridine), poly(acrylic acid), poly(methacrylic acid), poly(N-isopropylacrylamide), poly(N,N′-methylenbisacrylamide), poly(N-(N-propyl)acrylamide), poly(methacyclic acid), poly(2-hydroxyacrylamide), poly (ethylene glycol) acrylate, poly (ethylene glycol) methacrylate, and polysaccharides such as hyaluronic acid, dextran, alginate, agarose, and the like, and any co-polymer of the foregoing.

Hydrogel precursors (hydrogel-forming materials) forming such polymeric chains are contemplated, including any combination thereof.

Hydrogels are typically formed of, or are formed in the presence of, di- or tri- or multi-functional monomers, oligomer or polymers, which are collectively referred to as hydrogel precursors or hydrogel-forming agents or hydrogen-forming materials, having two, three or more polymerizable groups. The presence of more than one polymerizable group renders such precursors cross-linkable, and allow the formation of the three-dimensional network.

Exemplary cross-linkable monomers include, without limitation, the family of di- and triacrylates monomers, which have two or three polymerizable functionalities, one of which can be regarded as a cross-linkable functional group. Exemplary diacrylates monomers include, without limitation, methylene diacrylate, and the family of poly(ethylene glycol)ₙ dimethacrylate (nEGDMA). Exemplary triacrylates monomers include, without limitation, trimethylolpropane triacrylate, pentaerythritol triacrylate, tris (2-hydroxy ethyl) isocyanurate triacrylate, isocyanuric acid tris(2-acryloyloxyethyl) ester, ethoxylated trimethylolpropane triacrylate, pentaerythrityl triacrylate and glycerol triacrylate, phosphinylidynetris(oxyethylene) triacrylate.

In some of any of the embodiments described herein, a curable material, whether monomeric or oligomeric, can be a mono-functional curable material or a multi-functional curable material.

Exemplary polymers or co-polymers usable for forming a hydrogel according to the present embodiments include polyacrylates, polymethacrylates, polyacrylamides, polymethacrylamides, polyvinylpyrrolidone and copolymers of any of the foregoing. Other examples include polyethers, polyurethanes, and poly(ethylene glycol), functionalized by cross-linking groups or usable in combination with compatible cross linking agents.

Some specific, non-limiting examples, include: poly(2-vinylpiridine), poly(acrylic acid), poly(methacrylic acid), poly(N-isopropylacrylamide), poly(N,N′-methylenbisacrylamide), poly(N-(N-propyl)acrylamide), poly(methacyclic acid), poly(2-hydroxyacrylamide), poly (ethylene glycol) acrylate, poly (ethylene glycol) methacrylate, and polysaccharides such as dextran, alginate, agarose, and the like, and any co-polymer of the foregoing.

Hydrogel precursors (hydrogel-forming materials) forming such polymeric chains are contemplated, including any combination thereof.

Curable materials usable in the field of bioprinting are predominantly based on either naturally derived materials, including, for example, Matrigel, Alginate, Pectin, Xanthan gum, Gelatin, Chitosan, Fibrin, Cellulose and Hyaluronic acid, which can be isolated from animal or human tissues, or recombinantly-generated, or synthetically-prepared materials, including, for example, polyethyleneglycol; PEG, gelatin methacrylate; GelMA, poly(propylene oxide); PPO, poly(ethylene oxide); PEO; PEG, polyethyleneglycol-diacrylate, polyglutamic acid, gelatin methacrylate; GelMA, PLGA/PLLA, poly(dimethyl siloxane); Nanocellulose; Pluronic F127, short di-peptides (FF), Fmoc-peptide-based hydrogels such as Fmoc-FF—OH, Fmoc-FRGD-OH, Fmoc-RGDF-OH, Fmoc-2-Nal-OH, Fmoc-FG-OH, and thermoplastic polymers such as Polycaprolactone (PCL), Polylactic acid (PLA) or Poly(D,L-lactide-co-glycolide).

Exemplary curable materials usable in the context of the present embodiments include, but are not limited to, Matrigel, Gelatin methacrylate (GelMA), Nanocellulose (nano-scaled structured materials which are UV-curable, including cellulose nanocrystals (CNC), cellulose nanofibrils (CNF), and bacterial cellulose (BC), also referred to as microbial cellulose), Pluronic® materials, including, for example, Pluronic F127 which is fluid at a low temperature forms a gel at a high temperature, above critical micellar concentration (CMC) and Pluronic F127-diacrylate (DA) which is UV-curable, Hyaluronic acid (HA), Acrylated hyaluronic acid (AHA), methacrylated hyaluronic acid (MAHA), Poly-(ethylene glycol) diacrylate (PEGDA), Alginate, Xanthan gum, Pectin, Chitosan which can be crosslinked with a chemical agent such as Glutaraldehyde, Genipin or Sodium Tripolyphosphate (TPP).

Exemplary curable materials are described in the Examples section that follows.

Biological components or materials that can be included in one or more modeling material formulations as described herein include, cellular components, including, for example, culturing cells, and other cellular components such as cytokines, chemokines, growth factors; as well as other biological components such as proteins, agents that act to increase cell attachment, cell spreading, cell proliferation, cell differentiation and/or cell migration; an amino acid, peptides, polypeptides, proteins, DNA, RNA, lipids and/or proteoglycans.

Cells may comprise a heterogeneous population of cells or alternatively the cells may comprise a homogeneous population of cells. Such cells can be for example stem cells (such as embryonic stem cells, bone marrow stem cells, cord blood cells, mesenchymal stem cells, adult tissue stem cells), progenitor cells, or differentiated cells such as chondrocytes, osteoblasts, connective tissue cells (e.g., fibrocytes, fibroblasts and adipose cells), endothelial and epithelial cells. The cells may be naïve or genetically modified.

According to one embodiment of this aspect of the present invention, the cells are mammalian in origin.

Furthermore, the cells may be of autologous origin or non-autologous origin, such as postpartum-derived cells (as described in U.S. application Ser. Nos. 10/887,012 and 10/887,446). Typically the cells are selected according to the desired application.

Suitable proteins which can be used include, but are not limited to, extracellular matrix proteins [e.g., fibrinogen, collagen, fibronectin, vimentin, microtubule-associated protein 1D, Neurite outgrowth factor (NOF), bacterial cellulose (BC), laminin and gelatin], cell adhesion proteins [e.g., integrin, proteoglycan, glycosaminoglycan, laminin, intercellular adhesion molecule (ICAM) 1, N-CAM, cadherin, tenascin, gicerin, RGD peptide and nerve injury induced protein 2 (ninjurin2)], growth factors [epidermal growth factor, transforming growth factor-α, fibroblast growth factor-acidic, bone morphogenic protein, fibroblast growth factor-basic, erythropoietin, thrombopoietin, hepatocyte growth factor, insulin-like growth factor-I, insulin-like growth factor-II, Interferon-β, platelet-derived growth factor, Vascular Endothelial Growth Factor and angiopeptin], cytokines [e.g., M-CSF, IL-1beta, IL-8, beta-thromboglobulin, EMAP-II, G-CSF and IL-10], proteases [pepsin, low specificity chymotrypsin, high specificity chymotrypsin, trypsin, carboxypeptidases, aminopeptidases, proline-endopeptidase, Staphylococcus aureus V8 protease, Proteinase K (PK), aspartic protease, serine proteases, metalloproteases, ADAMTS17, tryptase-gamma, and matriptase-2] and protease substrates.

In addition, calcium phosphate materials, such as hydroxyapattite, for example, in a form of particles, can be used, including, but not limited to, nanoHA and nanoTCP. The particles size should be compatible with the dispensing heads so as to avoid clogging.

Non-curable materials, other than the biological materials as described herein, that can be included in one or more modeling formulations as described herein can be materials that impart a certain property to the formulation or to the hardened formulation and to the part of the object formed thereby. Such a property can be a physical property (e.g., an optical property such as transparency or opacity, color, a spectral property, heat resistance, electrical property and the like), or a mechanical or rheological property such as viscosity, elasticity, storage modulus, loss modulus, stiffness, hardness, and the like. Alternatively, or in addition, non-curable materials can be such that provide a biological function, for example, therapeutically active agents.

Exemplary non-curable materials include thixotropic agents, reinforcing agents, toughening agents, fillers, colorants, pigments, etc.

An exemplary non-curable material includes titanium dioxide.

An exemplary non-curable material includes oxidized cellulose.

According to some of any of the embodiments described herein, one or more of the modeling material formulations comprises hyaluronic acid.

According to some of any of the embodiments described herein, one or more of the modeling material formulations comprises hyaluronic acid featuring a curable group as defined herein.

According to some of any of the embodiments described herein, one or more of the modeling material formulations comprises one or more biological components or materials such as, but not limited to, cells, growth factors, peptides, heparan sulfate and fibronectin.

According to some of any of the embodiments described herein, one or more of the modeling material formulations comprises one or more agents that modify a mechanical property of the formulation and/or the object, as described herein, such as, but not limited to, alginate, hyaluronic acid, fibrinogen, elastin, peptides and a thixotropic agent (e.g., Crystaline nano cellulose (CNC)), oxidized cellulose, titanium dioxide, Clay mineral and carbon nanotubes.

In some of any of the embodiments described herein the rhCollagen-containing formulation further comprises one or more additional curable materials as described herein in any of the respective embodiments.

In some of these embodiments, a weight ratio of the curable recombinant human collagen and the additional curable material ranges from 10:1 to 1:2, or from 10:1 to 1:1, or from 5:1 to 2:1, including any intermediate value and subranges therebetween.

In some of any of the embodiments described herein the rhCollagen-containing formulation further comprises a thixotropic agent, as defined herein.

Herein throughout, the term “thixotropic” describes a property of a fluidic compound or material that is reflected by a time-dependent shear-thinning, that is its viscosity is decreased in correlation with the time at which shear forces are applied, and returns back to its original value when application of shear forces is ceased. In some of the present embodiments, a thixotropic material or agent is such that exhibits or imparts a significant, e.g., at least 100%, reduction in shear modulus under 50% strain.

In some of any of the embodiments described herein the rhCollagen-containing formulation further comprises a gel-forming agent, for example, a hydrogel-forming agent as described herein.

In some of any of the embodiments described herein the rhCollagen-containing formulation further comprises a biological component or material as described herein.

In some of any of the embodiments described herein the rhCollagen-containing formulation further comprises one or more curable or non-curable materials as described herein in any of the respective embodiments.

According to some of any of the embodiments described herein the recombinant human collagen is selected from a monomeric collagen and a fibrillar collagen, as exemplified in the Examples section that follows.

According to some of any of the embodiments described herein, the mol ratio of the curable group(s) and the collagen is essentially as exemplified in the Examples section herein.

According to some of any of the embodiments described herein the formulation further comprises hyaluronic acid, as described herein.

According to some of any of the embodiments described herein the formulation further comprises hyaluronic acid featuring a polymerizable group.

According to some of any of the embodiments described herein the formulation further comprises one or more biological components such as, but not limited to, a component selected from cells, growth factors, peptides, heparan sulfate and fibronectin.

According to some of any of the embodiments described herein the formulation further comprises one or more agents that modify a mechanical property of the formulation and/or the object, such as, but not limited to, alginate, hyaluronic acid, fibrinogen, elastin, peptides and a thixotropic agent (e.g., Crystaline nano cellulose (CNC)).

According to some of any of the embodiments described herein the formulation features a neutral pH (e.g., from about 6 to about 8).

According to some of any of the embodiments described herein the formulation features viscosity parameters essentially as described herein.

According to some of any of the embodiments described herein the formulation features approximately the same viscosity at 4° C. and at a temperature above 10, or above 20° C. (e.g., 37° C.).

In embodiments where two or more modeling material formulations are used, two of more formulations are rh-Collagen containing formulations as described herein, which differ from one another by the presence, type and/or concentration of an additional material that is included therein. For example, one formulation can comprise curable rhCollagen, and another formulation can comprise curable rhCollagen and a biological material as described herein. For example, one formulation can comprise curable rhCollagen, and another formulation can comprise curable rhCollagen and an additional curable material as described herein. For example, one formulation can comprise curable rhCollagen and one additional curable material, and another formulation can comprise curable rhCollagen and another additional curable material as described herein. For example, one formulation can comprise curable rhCollagen and one additional curable material, and another formulation can comprise curable rhCollagen and a non-curable material as described herein. Any other combinations are contemplated.

In some of any of the embodiments described herein, all the curable materials in the building material are cured under the same curing condition. In some embodiments, all curable materials are photocurable.

In some of any of the embodiments described herein, a modeling material formulation that comprises a curable material further comprises an agent that promotes curing or hardening of the curable material when exposed to a curing condition.

The concentration of the agent can be determined in accordance with the concentration of the curable material and the desired degree of curing (e.g., desired cross-linking degree).

When the curable materials are photocurable materials, the agent is a photoinitiator. The photoinitiator is selected in accordance with the curing mechanism (e.g., free-radical, cationic, etc.).

A free-radical photoinitiator may be any compound that produces a free radical on exposure to radiation such as ultraviolet or visible radiation and thereby initiates a polymerization reaction. Non-limiting examples of suitable photoinitiators include benzophenones (aromatic ketones) such as benzophenone, methyl benzophenone, Michler's ketone and xanthones; acylphosphine oxide type photo-initiators such as 2,4,6-trimethylbenzolydiphenyl phosphine oxide (TMPO), 2,4,6-trimethylbenzoylethoxyphenyl phosphine oxide (TEPO), and bisacylphosphine oxides (BAPO's); benzoins and bezoin alkyl ethers such as benzoin, benzoin methyl ether and benzoin isopropyl ether and the like. Examples of photoinitiators are alpha-amino ketone, and bisacylphosphine oxide (BAPO's).

Exemplary photoinitiators include, but are not limited to, those of the Irgacure® family, riboflavin, rose Bengal, and more.

A free-radical photo-initiator may be used alone or in combination with a co-initiator. Co-initiators are used with initiators that need a second molecule to produce a radical that is active in the photocurable free-radical systems. Benzophenone is an example of a photoinitiator that requires a second molecule, such as an amine, to produce a free radical. After absorbing radiation, benzophenone reacts with a ternary amine by hydrogen abstraction, to generate an alpha-amino radical which initiates polymerization of acrylates. Non-limiting example of a class of co-initiators are alkanolamines such as triethylamine, methyldiethanolamine and triethanolamine.

Suitable cationic photoinitiators include, for example, compounds which form aprotic acids or Bronsted acids upon exposure to ultraviolet and/or visible light sufficient to initiate polymerization. The photoinitiator used may be a single compound, a mixture of two or more active compounds, or a combination of two or more different compounds, i.e. co-initiators. Non-limiting examples of suitable cationic photoinitiators include aryldiazonium salts, diaryliodonium salts, triarylsulphonium salts, triarylselenonium salts and the like. An exemplary cationic photoinitiator is a mixture of triarylsolfonium hexafluoroantimonate salts.

Non-limiting examples of suitable cationic photoinitiators include P-(octyloxyphenyl) phenyliodonium hexafluoroantimonate UVACURE 1600 from Cytec Company (USA), iodonium (4-methylphenyl)(4-(2-methylpropyl)phenyl)-hexafluorophosphate known as Irgacure 250 or Irgacure 270 available from Ciba Speciality Chemicals (Switzerland), mixed arylsulfonium hexafluoroantimonate salts known as UVI 6976 and 6992 available from Lambson Fine Chemicals (England), diaryliodonium hexafluoroantimonate known as PC 2506 available from Polyset Company (USA), (tolylcumyl) iodonium tetrakis (pentafluorophenyl) borate known as Rhodorsil® Photoinitiator 2074 available from Bluestar Silicones (USA), iodonium bis(4-dodecylphenyl)-(OC-6-11)-hexafluoro antimonate known as Tego PC 1466 from Evonik Industries AG (Germany).

### The Object:

Herein throughout, in the context of bioprinting, the term “object” describes a final product of the additive manufacturing which comprises, in at least a portion thereof, a biological component. This term refers to the product obtained by a bioprinting method as described herein, after removal of the support material, if such has been used as part of the uncured building material.

The term “object” as used herein throughout refers to a whole object or a part thereof.

In the context of the present embodiments, the object comprises in at least a portion thereof a collagen-based material.

By “collagen-based material” it is meant a material that comprises collagen, preferably a recombinant human collagen as described herein in any of the respective embodiments and any combination thereof.

In some of any of the embodiments described herein, the collagen-based material comprises a scaffold, for example, a hydrogel scaffold, made of a three-dimensional fibrillar network that comprises recombinant human collagen as described herein.

In some of any of the embodiments described herein, the collagen-based material comprises polymerized and/or cross-linked recombinant human collagen, in which a plurality of monomeric and/or fibrillar collagen units are linked to one another to thereby form a three-dimensional network.

The three-dimension network or scaffold can be in a form of, for example, a film, a sponge, a porous structure, a hydrogel, and any other form, according to a desired need.

In some of any of the embodiments described herein, the object is in a form of a tissue or organ, which comprises, in at least a portion thereof, a collagen-based material as described herein. Such an object can be formulated in accordance with a respective 3D printing data of a desired organ or tissue, using, in addition to the curable collagen as described herein, additional curable materials and biological materials as described herein.

In some embodiments, the object is an implantable object. In some embodiments, the object is an artificial skin. In some embodiments, the object is an artificial tissue (e.g., connective tissue, or muscle tissue such as cardiac tissue and pancreatic tissue). Examples of connective tissues include, but are not limited to, cartilage (including, elastic, hyaline, and fibrocartilage), adipose tissue, reticular connective tissue, embryonic connective tissues (including mesenchymal connective tissue and mucous connective tissue), tendons, ligaments, and bone.

In some embodiments, the object is usable in, or is for use in, constructing an artificial organ or tissue.

The object can further comprise hardened materials formed of one or more of the additional curable materials as described herein in any of the respective embodiments, biological components or materials, as described herein in any of the respective embodiments, and/or non-curable materials as described herein in any of the respective embodiments.

In some embodiments, the object is in a form of a collagen scaffold or film, that can be used in research or therapeutic applications, for example, in repairing a damaged tissue, for example, upon seeding culturing cells therein, or in wound healing.

The scaffolds may be administered to subjects in need thereof for the regeneration of tissue such as connective tissue, muscle tissue such as cardiac tissue and pancreatic tissue.

The films can be used to construct biomedical devices such as, for example, collagen membranes for hemodialysis.

According to some embodiments, films or scaffolds can be used in cell cultures.

The phrase “cell culture” or “culture” as used herein refers to the maintenance of cells in an artificial, e.g., an in vitro environment. It is to be understood, however, that the term “cell culture” is a generic term and may be used to encompass the cultivation not only of individual prokaryotic (e.g., bacterial) or eukaryotic (e.g., animal, plant and fungal) cells, but also of tissues, organs, organ systems or whole organisms.

In some embodiments, the films or scaffolds can be used in a wound healing process.

In some embodiments, collagen films provided herein are used to prevent adhesions following tendon injuries, to lengthen levator palpebrae muscles ophthalmic surgery, and to repair transected nerves. Collagen films provided herein may further be used for burn dressings and in healing of bone defects.

The object of the present embodiments comprises a myriad of other uses including, but not limited to, in the treatment of diseases such as interstitial cystitis, scleroderma, and rheumatoid arthritis cosmetic surgery, as a healing aid for burn patients, as a wound-healing agent, as a dermal filler, for spinal fusion procedures, for urethral bulking, in duraplasty procedures, for reconstruction of bone and a wide variety of dental, orthopedic and surgical purposes.

### Kits:

According to an aspect of some embodiments of the present invention, there is provided a kit that comprises a curable rhCollagen as described herein in any of the respective embodiments.

According to some embodiments, the kit comprises a composition that comprises curable rhCollagen as described herein in any of the respective embodiments.

According to some embodiments, the composition comprises the curable rhCollagen in a lyophilized form.

According to some embodiments, the composition comprises the curable rhCollagen and an aqueous solution or carrier.

According to some of any of the embodiments described herein, the kit is identified for use, or is usable, as a modeling material formulation for additive manufacturing (e.g., bioprinting) of an object as described herein in any of the respective embodiments.

According to some of any of the embodiments described herein, the kit further comprises an aqueous carrier, as described herein in any of the respective embodiments. In some embodiments, the composition and the aqueous carrier are packaged individually within the kit.

Alternatively, the kit includes instructions to prepare a modeling material formulation as described herein, by mixing the composition with the aqueous carrier.

It is expected that during the life of a patent maturing from this application many relevant curable biocompatible materials, bioprinting media, and/or bioprinting technologies will be developed and the scope of embodiments related to curable biocompatible materials, bioprinting media, and/or bioprinting technologies is intended to include all such new technologies a priori.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Materials

Human recombinant collagen (rhCollagen) type I expressed and isolated from transgenic tobacco plants was produced and supplied by CollPlant Ltd (Israel).

Type I Bovine Collagen (PureCol®) and Bovine-based Methacrylated collagen were purchased from Advanced Biomatrix, USA.

Methacrylic anhydride, glycidyl methacrylate, triethylamine, tetrabutylammonium bromide, 2,4,6-Trinitrobenzenesulfonic acid (TNBS), 2-Hydroxy-4′-(2-hydroxyethoxy)-2-methylpropiophenone (Irgacure 2959), sodium phosphate monobasic anhydrous, HCl 1N, HCl>37%, sodium bicarbonate, Titanium dioxide (TiO₂), Hepes Buffer, Eosin Y, N-Vinylpyrrolidone and NaOH were purchased from Sigma Aldrich Ltd, Israel.

Dulbaco Phosphate buffer saline (DPBS), X10 DPBS, Foetal Bovine Serum (FBS), DMEM high glucose and penicilin/streptomicin were purchased from Biological Industries Ltd, Israel.

Sodium phosphate dibasic anhydrous was purchased from Canton, India.

Ethanol absolute and acetone were purchased from Bio-Lab Ltd, Israel.

Hyaluronic acid was purchased by Lifecore, USA.

Acetic acid was purchased from JT Baker or Advanced Biometrix.

Oxidized cellulose was received from Synthesia.

PVAMA (polyvinyl alcohol methacrylate) was provided by Prof. Shoseyov.

TEA (triethanolamine) was purchased from J. T. Baker

Nano-hydroxyapatite (nanoHA) and Nano-tricalcium phosphate (nanoTCP) were purchased from Fluidinova.

### Buffer Stock Solutions Preparation:

Fibrillogenesis buffer (FB): sodium phosphate dibasic was dissolved in double distilled water (DDW) to a final concentration of 162 mM. The solution was titrated to pH 11.2 with 10N NaOH.

Medium preparation: 50 mL of fetal bovine serum and 5 mL of penicillin/streptomycin (10,000 units/mL and 10 mg/mL respectively) were added under aseptic conditions to 500 mL of DMEM high glucose medium. The medium was gently mixed and kept in fridge.

Phosphate Buffer (0.1 M)/Saline (0.016 M) preparation (300 mOs/L): 1 M Phosphate buffer was prepared as follows: 31.6 mL of 1 M sodium phosphate dibasic were mixed with 68.4 mL of 1 M sodium phosphate monobasic. The obtained 1 M Phosphate Buffer was diluted to 0.1 M by adding DDW (9:1, vol/vol). NaCl was added to a final concentration of 15.8 mM to achieve physiological osmolarity (300 mOs/L).

Phosphate buffer (0.1 M: pH 7.4)/Saline (11.3 mM) (physiological osmolarity) preparation: 1 M Phosphate buffer was prepared as follows: 77.4 mL of 1 M sodium phosphate dibasic were mixed with 22.6 mL of 1 M sodium phosphate monobasic. The obtained 1 M Phosphate Buffer was diluted to 0.1 M by adding DDW (9:1, vol/vol). NaCl was added to a final concentration of 11.3 mM to achieve physiological osmolarity (300 mOs/L).

Washing buffer: HCl was added to the fibrillogenesis buffer to reach a final concentration of 16.2 mM sodium phosphate dibasic and 10 mM HCl. pH was adjusted to 7.2-7.4 with 10N NaOH.

### Instrumental Data:

¹H-NMR spectra were recorded at 25° C. on a Bruker Avance II spectrometer, operating at a 1H resonance frequency of 500 MHz, using D₂O as a solvent. The analysis was quantitative NMR using 64 scans for signal-to-noise averaging, and a recycle delay of 8.6 sec. Values provided represent the δ (normalized chemical shift) in ppm.

Viscosity and G′/G″ measurements were performed on a HAAKE RheoStress600 rheometer (Thermo Electron Corporation) with a temperature-controlled cell chamber, using a C60/1°Ti or C35/1°Ti cone-plate set ups. Analysis of cross-linked (hardened) discs was performed using PP20 serrated spindle and 20 mm serrated plate set up.

In-situ crosslinked experiments were conducted on TA discovery hybrid temperature controlled rheometer with a UV chamber.

Other measurements were performed as described in the following.

### Example 1

Methacrylation of rhCollagen

Fibrillar rhCollagen-methacrylamide and monomeric rhCollagen-methacrylamide were prepared by reaction of lysine and hydroxylysine residues of the collagen with methacrylic anhydride in aqueous medium as described below, and stored at 4° C. light protected until further use.

Fibrillar rhCollagen-Methacrylamide:

3 to 10 mg/mL fibrillar rhCollagen-methacrylamide (rhCollagen-MA) was synthesized either in a 1× washing buffer as described herein, a fibrillogenesis buffer as described herein or DDW, at room temperature (R.T.) or at 12° C.

In an exemplary procedure, fibrillar rhCollagen-MA was synthesized in DDW as follow: monomeric rhCollagen 3-4 mg/mL solution in 10 mM HCl, (Collage™) was mixed with fibrillogenesis buffer at 9:1 v/v ratio and stirred for 1 hour at R.T, receiving fibrils. The solution was centrifuged at 7500 rpm at 4° C. for 30 minutes, and the supernatant was thereafter discarded. The remaining pellet was re-suspended in equal volume of washing buffer and centrifuged at the same conditions. Thereafter, the sediment fibrils were re-suspended in DDW to obtain a 10 mg/mL solution. The concentration was confirmed by percent solid measurements. Methacrylic anhydride was added drop-wise under nitrogen flow at room temperature at 10 to 20 molar ratio with respect to collagen lysines (10-20 fold), and the reaction solution pH was monitored over time and adjusted to pH 7 with 10N NaOH. After 24 hours reaction, the mixture was dialyzed against washing buffer (pH=7) using 100 kDa cutoff dialysis tubing (Spectrum Laboratories Inc, CA, US) for 3 days at 4° C. with at least 6 changes of the dialysate (washing buffer in this case), to remove reaction by-products, and was thereafter lyophilized for 3-4 days.

The extent of modification of rhCollagen was quantified using TNBS (2,4,6-Trinitrobenzenesulfonic acid) colorimetric assay. This assay quantifies the molar content of free, non-reacted ε-amino groups derived from lysine and hydroxyl lysine, and accordingly the degree of functionalization.

The assay was conducted in accordance with a previously described protocol [Sashidhar et al., Journal of Immunological Methods. 1994, 167, 121-127], and based on Habeeb [Habeeb A.F.S.A, Analytical Biochemistry. 1966, 14, 328-336]. Briefly, freshly prepared 0.4 mL of 0.01% (v/v) TNBS was added to 0.4 mL of 0.1-2 mg/mL fibrillar rhCollagen-MA in sodium bicarbonate 4%. After 2 hours reaction at 40° C., 0.2 mL of 1N HCl and 0.4 mL of 10% (v/v) SDS were added. The absorbance was measured at 335 nm in a spectrophotometer in a 1 mL polystyrene cuvette. A control (blank) was prepared with the same procedure except that sodium bicarbonate buffer was added instead of rhCollagen-MA solution. The absorption of 1-2 mg/mL native fibrillar rhCollagen prepared with the same conditions was recorded for calibration.

Table 1 below presents the data obtained for the degree of functionalization for batches obtained upon reacting the fibrillar rhCollagen with 10-folds, 15-folds or 20-folds mol ratio of methacrylic anhydride, as determined by the TNBS assay.

TABLE 1

| MA:Fibrillar | Degree of |
|---|---|
| rhCollagen mol ratio | methacrylation [%] |
| 10:1 | 98.1 |
| 15:1 | 95.5 |
| 20:1 | 92.9 |

These results demonstrate the high modification of the fibrillar rhCollagen at all MA concentrations, and suggest that adding the methacrylic reagent at a molar ratio of 10:1 may be preferable for receiving maximal functionalization of the fibrillar rhCollagen.

Monomeric rhCollagen-Methacrylamide:

Monomeric rhCollagen-methacrylamide was synthesized while using 200 mM of MOPS, phosphate, or Tris buffer with the addition of 150 mM NaCl.

In an exemplary procedure, 200 mM MOPS and 150 mM NaCl were added to 3-4 mg/mL Collage™ (a solution of Monomeric rhCollagen in 10 mM HCl and the solution was stirred at room temperature until clear solution was obtained. Thereafter, 1 to 20-fold molar ratio excess of methacrylic anhydride was added drop-wise under nitrogen flow at 12° C., and the pH was adjusted over time to pH 7 with 10N NaOH. After 24 hours reaction, the mixture was initially dialyzed against 10 mM HCl/20 mM NaCl (pH=2) with 100 kDa cutoff dialysis hollow fiber cartridge (GE Healthcare), at least 5 changes of the dialysate, followed by dialysis against 10 mM HCl. The material was then lyophilized.

The structural properties of non-modified monomeric rhCollagen and the degree of methacrylation of collagen lysine amines of rhCollagen (also referred to herein as Coll-MA) were analyzed by ¹H-NMR spectroscopy (500 MHz). Representative spectra are shown in FIG. 1 for Coll-MA synthesized with 10-folds molar excess of methacrylic anhydride. The signals of the methyl function and acrylic protons of the introduced methacrylate are indicated as (b) and (c), respectively; the lysine methylene signals of non-modified rhCollagen as (a).

The spectrum of the non-modified rhCollagen was in high agreement with collagen spectra reported in the literature [R. Ravichandran et. al; J. Mater. Chem. B, 2016, 4, 318-326].

The degree of methacrylation can be calculated from the obtained spectrum using a quantitative analysis technique. The signal at δ=3 ppm (a) is ascribed to the lysine methylene groups in Collagen, and was significantly reduced after methacrylation. Compared to non-modified rhCollagen, new signals can be observed at 1.9 ppm (b) and 5.4-5.7 ppm (c) in the spectrum of Coll-MA, which are assigned to the methyl function and the acrylic protons of the introduced methacrylic groups, respectively, confirming a successful attachment of the methacrylate to the collagen.

### Example 2

Viscosity Measurements of rh-Collagen and rh-Collagen-MA

### Solutions Preparation for Viscosity Measurements:

PureCol® and Collage™ in DPBS: 8 ml of monomeric collagen solutions (3 mg/mL in 10 mM HCl), either rhCollagen (Collage™) or bovine collagen (PureCol®) were neutralized by adding 1 mL of DPBS ×10. The solution was then brought to pH 7-7.5 by titration with 0.1N NaOH. Double distillated water (DDW) was then added to reach a final volume of 10 mL. Samples were incubated at 37° C. for at least 90 minutes before measurements were performed (either at 37° C. or 4° C.).

Collage™ in fibrillogenesis buffer: 9 ml of monomeric rhCollagen (Collage™) solution (3.79 mg/mL in 10 mM HCl) was neutralized by adding 1 mL of fibrillogenesis buffer. Samples were incubated at 37° C. for at least 90 minutes before measurements were performed (either at 37° C. or 4° C.).

Fibrillar rhCollagen-methacrylamide in DPBS: Lyophilized fibrillar rhCollagen-MA prepared in DDW and dialyzed vs. washing buffer (as described in Example 1 hereinabove, using 10:1 molar ratio of MA:rhCollagen) was dissolved in DPBS to a concentration of 10 mg/mL. Samples were incubated at 37° C. for at least 90 minutes before measurements were performed (either at 37° C. or 4° C.).

Monomeric rhCollagen-methacrylamide in acetic acid: 10 mg/ml rhCollagen-methacrylamide solution in 20 mM Acetic acid was prepared by adding the appropriate medium volume to the lyophilized material and mixing at room temperature until receiving complete dissolution.

Bovine collagen-methacrylate in acetic acid: 10 mg/ml Bovine collagen-methacrylate solution in 20 mM Acetic acid was prepared by adding the appropriate medium volume (Advanced BioMatrix) to the lyophilized material and mixing at room temperature until receiving complete dissolution.

Temperature Dependence of rhCollagen/Bovine Collagen Viscosity:

FIG. 2 presents comparative plots showing the viscosity of rhCollagen (Collage™) and Bovine Collagen (PureCol) in DPBS expressed as a function of shear rate at T=4° C. and T=37° C. Bovine collagen (solid lines) shows clear temperature dependence of the zero shear rate viscosity (η₀), i.e., the viscosity plateau at low shear rate values, having at 37° C. η₀ values that are more than one order of magnitude higher than the values at 4° C. On the contrary, rhCollagen (dashed lines) shows no significant difference between η₀ values at 4° C. and 37° C.

FIG. 3 presents comparative plots showing the viscosity of rhCollagen neutralized in FB, prepared as described hereinabove, and indicate a very similar behavior, i.e., the viscosity at 4° C. and 37° C. is almost identical, at all shear rates.

Viscosity of rhCollagen-Methacrylamide:

FIG. 4 presents comparative plots showing the viscosity of fibrillar rhCollagen-MA in DPBS at 4° C. (solid line) and 37° C. (dashed line), and indicate that the profiles are similar (both are shear thinning) but not identical, yet, the zero shear rate values are around 1000 cP at both temperatures.

FIG. 5 presents comparative plots further demonstrating the thermal stability and, importantly, the decrease in viscosity within a range of increased working (e.g., jetting) temperatures, compared with an increase in viscosity as the temperature increases shown for bovine-based methacrylated curable collagen. Viscosity measurements were performed with increased temperature steps, for 10 mg/ml rhCollagen-MA solution in 20 mM acetic acid (white circles) and for bovine-based curable collagen in acetic acid (black circles), as recorded in the rheometer using Rotational temperature steps CR mode with 5 [1/sec] rate. The acetic acid solutions were prepared as described hereinabove.

### Example 3

Characterization of Hardened rh-Collagen-MA
rhCollagen-MA Photocrosslinking for Loss and Storage Moduli Measurements:

rhCollagen-MA crosslinked scaffolds were formed in two different preparations, aimed to be examined in two individual experiments, respectively.

A photoinitiator 10% (v/v) stock solution was prepared as follows: Irgacure 2959 was dissolved in ethanol absolute/DDW 1:1 solution to a final concentration of 100 mg/mL.

In the first preparation, 1-2 wt % fibrillar rhCollagen-MA synthesized with 10-fold molar excess of the methacrylic reagent were dissolved in DPBS 0.1 M at room temperature, then Irgacure 2959 0.1% was added into 1 mL final volume of solution replicates which were injected into a discoid mold. Thereafter, curing process was performed from a distance of 1.5 cm for 7 and 10 seconds at an averaged intensity of 670 mW/cm² using mercury light source, resulting in cross-linked scaffolds.

The second preparation included 2 different batches of fibrillar rhCollagen-MA, synthesized with 15-fold and 20-fold molar excess of the methacrylic reagent. 1-2 wt % were dissolved in DPBS 0.1 M, and Irgacure 2959 0.1% was added to achieve a final volume of 1.5 mL. In order to obtain highly crosslinked scaffolds, curing process was performed from a distance of 2 cm for 60 seconds at an averaged intensity of 420 mW/cm², using mercury light source.

### Scaffolds' Storage and Loss Moduli Measurements:

The rheological behavior of rhCollagen crosslinked discs was investigated using parallel plate system employing PP20 serrated spindle and 20 mm serrate plate set up. Characterization of the non-crosslinked rhCollagen-MA was performed using C60/1°Ti cone-plate elements.

In order to evaluate the rheological behavior of rhCollagen-MA, two sets of experiments were performed individually.

In the first set, oscillation over time analysis was conducted at 37° C. and is presented in FIGS. 6-A and 6-B. 1 mL samples were subjected to oscillation forces at controlled stress mode, while applying 5 Pa shear stress at 1 Hz frequency and 37° C. for 300 seconds. FIG. 6A presents loss/storage moduli (G′/G″) and the tan (delta) values before UV curing, and FIG. 6B presents these values following exposure to light curing (following curing by cross-linking upon addition of a photoinitiator). The cured samples, 1 ml discs, were tested as the gap was adjusted to 90% of the original sample height. G′ and G″ values were averaged at the range of 150-300 seconds in all measurements.

The results demonstrate that the storage modulus of the rhCollagen-MA increases by 2-folds upon illumination (curing by irradiation) in the presence of a photoinitiator. Moreover, the results demonstrate the ability to control the scaffold properties by changing the rhCollagen-MA concentration and time of illumination (irradiation; exposure to a curing condition). High differences between G′ and G″ values, and close-to-zero tan (delta) values of the cross-linked discs indicate an elastic-like behavior.

In the second set, 1.5 mL cured (cross-linked) discs were characterized by applying oscillations under frequency sweep at 37° C. G′ was recorded under 1 Pa shear stress at frequency range of 0.01-100 Hz. To initiate measurement, the spindle was lowered to contact the hydrogel surface, and then further lowered until the axial force of the instrument was equal to 0.4 N.

Prior to all measurements, samples were kept on the plate covered with humidity lid for 1 minute, in order to reach temperature equilibrium.

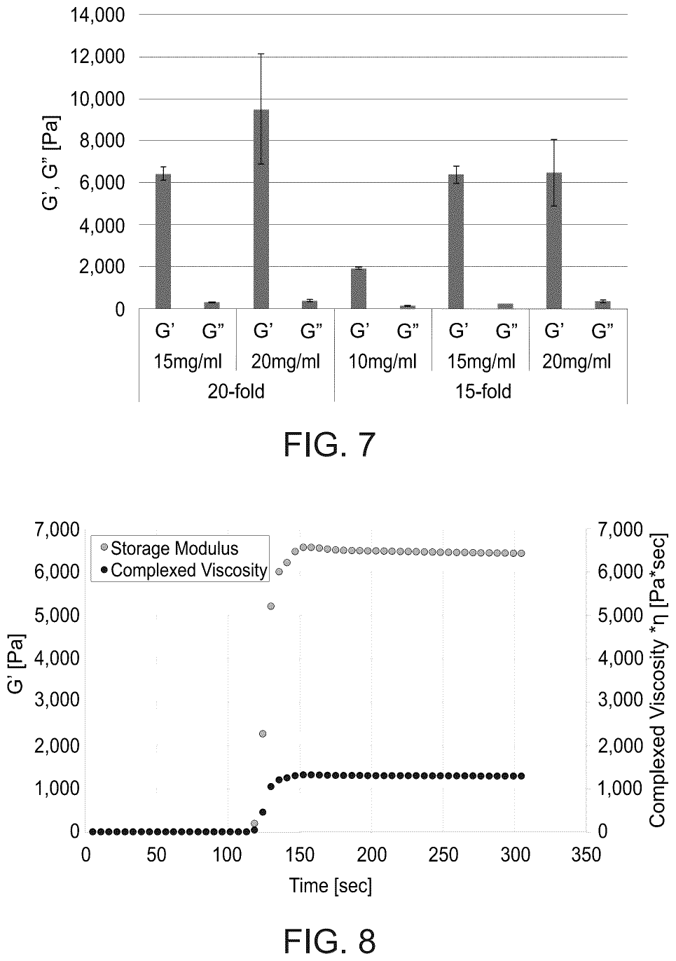

The results are presented in FIG. 7, and show that 1.5 mL discs illuminated for 60 seconds feature higher G′ values. G′ increases with increasing rhCollagen-MA concentration and increasing degree of methacrylation, indicating the ability to control the scaffolds properties.

In Situ Crosslinking of rhCollagen-MA:

A solution of 15 mg/ml monomeric rhCollagen-MA in 10 mM HCl was prepared by adding the appropriate volume of the medium to the lyophilized material, and after full dissolution, 0.1% of Irgacure photoinitiator was added to each replicate.

In situ crosslinking of rhCollagen-MA was tested by applying oscillation forces over time at 25° C. using 7% strain and angular frequency of 5 rad/sec. 80 μl solution was placed in 20 mm parallel plate geometry, and G′ and complexed viscosity properties were first measured prior to cross-linking as described hereinabove. Thereafter an external UV 365 nm light source was introduced, and the properties of the sample were recorded over time during the cross-linking reaction on the plate.

The results are presented in FIG. 8. As shown therein, an immediate sharp increase in the tested properties was observed during a short period, reaching a plateau after less than 30 seconds, indicating a good curability of the formulation. After 60 seconds curing, light source was off and values were kept measured post curing over time, showing similar values and indicating stability.

### Example 4

Characterization of rhCollagen-MA Formulations in Varying Mediums and/or with Varying Additives

### Preparation of DMEM Formulations:

rhCollagen-methacrylamide in DMEM: Lyophilized fibrillar rhCollagen-methacrylamide (prepared as described in Example 1 hereinabove, using with 15:1 molar ratio of MA:rhCollagen) was dissolved in DMEM medium to final concentrations of 20 and 26 mg/mL.

rhCollagen-methacrylamide/Hyaluronic Acid in DMEM: Hyaluronic Acid was added to a solution of fibrillar rhCollagen-MA to obtain final concentrations of 10 mg/mL HA and 20 mg/mL rhCollagen-MA in DMEM medium.

### Preparation of Hyaluronic Acid Methacrylate (HAMA):

500 mg of HA were functionalized as described by Leach et al. [Leach et.al. 2002, Biotechnology and Bioengineering, vol. 82, no. 5]. Briefly, 1.8 mL of triethylamine, 1.8 mL of glycidyl methacrylate, and 1.8 grams of tetrabutyl ammonium bromide were added separately to 50 mL of 10 mg/mL HA solution in DDW, while thoroughly mixing the reaction mixture before the addition of each component. The obtained reaction mixture was stirred overnight at room temperature, and thereafter the HAMA was precipitated by adding 20-folds volume of acetone and was then re-dissolved in DDW. The precipitation process was repeated twice to eliminate all possible side products, and the obtained reaction product was then lyophilized.

rhCollagen-methacrylamide/Hyaluronic Acid methacrylate (HAMA) in DMEM: Hyaluronic Acid methacrylate prepared as described hereinabove was added to a solution of fibrillar rhCollagen-MA to obtain final concentrations of 10 mg/mL HA-MA and 20 mg/mL rhCollagen-MA in DMEM medium.

### Viscosity of DMEM Formulations:

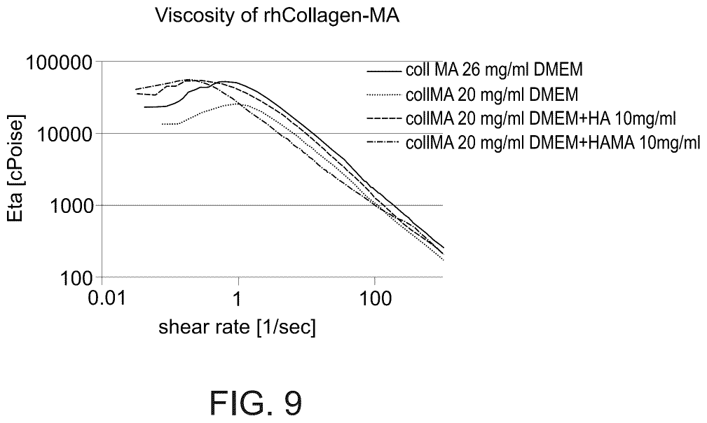

The viscosity of rhCollagen-MA in DMEM at 25° C., alone and in the presence of hyaluronic acid (HA) or HA-MA (prepared as described in Example 1) was tested, and the obtained data is presented in FIG. 9.

As can be seen therein, the typical shear thinning behavior of the rhCollagen shown in FIGS. 2 and 3 is maintained also for the rhCollagen-MA with and without the addition of HA/HA-MA. Upon increasing the concentration of rhCollagen from 20 to 26 mg/mL the zero shear viscosity increases, and the same is observed upon the addition of 10 mg/mL HA or HAMA (which leads to final polymer concentration of 30 mg/mL).

### Preparation of Formulations in 0.1 M Phosphate Buffer 0.1 M (pH 7.4)/NaCl 11.3 mM:

Stock solutions of 7.5 mg/ml monomeric rhCollagen-methacrylamide (10 fold) in Phosphate Buffer (0.1 M)/Saline (11.3 mM), 30 mg/ml PVAMA in Phosphate Buffer (0.1 M)/Saline (11.3 mM), 30 mg/ml TiO₂ in Phosphate Buffer (0.1 M)/Saline (11.3 mM), 30 mg/ml Oxidized Cellulose in Phosphate Buffer (0.1 M)/Saline (11.3 mM) were prepared. HAMA was purchased from Advanced BioMatrix and dissolved to 10 mg/ml in 10 mM HCl. 4.5 ml of HAMA solution were eventually neutralized by adding 0.5 ml of 1 M Phosphate buffer+113 mM NaCl.

Preparation of monomeric rhCollagen-methacrylamide in Phosphate Buffer (0.1 M)/Saline (11.3 mM): Stock solution of monomeric rhCollagen methacrylamide was dilute to 5 mg/ml with 0.1 M Phosphate Buffer (pH7.4)+11.3 mM NaCl

Preparation of rhCollagen-methacrylamide and HAMA in Phosphate Buffer (0.1 M)/Saline (11.3 mM): Solutions of CollagenMA (monomeric rhCollagen-methacrylamide) and HAMA in a weight ratio of collagenMA:HAMA 5:1 and 2:1 were prepared by mixing the appropriate amount of the collagenMA and HAMA stock solutions described herein above with Phosphate Buffer (0.1 M)/Saline (11.3 mM) to obtain final concentrations of 5 mg/ml CollMA and 1 mg/ml HAMA (5:1 ratio) and 5 mg/ml coll MA and 2.5 mg/ml HAMA (2:1 ratio), respectively.

Preparation of rhCollagen-methacrylamide and PVAMA in Phosphate Buffer (0.1 M)/Saline (11.3 mM): CollagenMA (monomeric rhCollagen-methacrylamide) and PVAMA in a weight ratio of collagenMA:PVAMA 5:1, 2:1 and 1:2 were prepared by mixing the appropriate amount of the collagenMA and PVAMA stock solutions described herein above with Phosphate Buffer (0.1 M)/Saline (11.3 mM) to obtain final concentrations of 5 mg/ml CollMA and 1 mg/ml PVAMA (5:1 ratio), 5 mg/ml collMA and 2.5 mg/ml PVAMA (2:1 ratio) and 5 mg/ml CollMA and 10 mg/ml PVAMA (1:2 ratio),

Preparation of rhCollagen-methacrylamide and Titanium Dioxide (TiO₂) in Phosphate Buffer (0.1 M)/Saline (11.3 mM): CollagenMA (monomeric rhCollagen-methacrylamide) and Titanium Dioxide (TiO₂) in a weight ratio of collagenMA:Titanium Dioxide (TiO₂) of 5:1, 2:1 and 1:2 were prepared by mixing the appropriate amount of the collagenMA and Titanium Dioxide (TiO₂) stock solutions described herein above with Phosphate Buffer (0.1 M)/Saline (11.3 mM) to obtain final concentrations of 5 mg/ml CollMA and 1 mg/ml Titanium Dioxide (TiO₂) (5:1 ratio), 5 mg/ml collMA and 2.5 mg/ml Titanium Dioxide (TiO₂) (2:1 ratio) and 5 mg/ml CollMA and 10 mg/ml Titanium Dioxide (TiO₂) (1:2 ratio).

Preparation of rhCollagen-methacrylamide and oxidized cellulose in Phosphate Buffer (0.1 M)/Saline (11.3 mM): CollagenMA (monomeric rhCollagen-methacrylamide) and oxidized cellulose in a weight ratio of collagenMA:oxidized cellulose of 5:1, 2:1 and 1:2 were prepared by mixing the appropriate amount of the collagenMA and oxidized cellulose stock solutions described hereinabove with Phosphate Buffer (0.1 M)/Saline (11.3 mM) to obtain final concentrations of 5 mg/ml CollMA and 1 mg/ml oxidized cellulose (5:1 ratio), 5 mg/ml collMA and 2.5 mg/ml oxidized cellulose (2:1 ratio) and 5 mg/ml CollMA and 10 mg/ml oxidized cellulose (1:2 ratio).

### Viscosity of Formulations in 0.1 M Phosphate Buffer 0.1 M (pH 7.4)/NaCl 11.3 mM:

The viscosity of formulations containing 5 mg/ml rhCollagen methacrylate (rhCollagen-MA; col) enriched with different additives (polivinyl alcohol methacrylate (PVAMA), hyaluronic acid methacrylate (HAMA), Titaniud DiOxide (TiO2) and oxidized cellulose (OC)) at collagenMA:additive ratios of 5:1, 2:1 and 1:2, in 0.1 M Phosphate buffer pH 7.4/11.3 mM NaCl (physiological osmolarity), prepared as described herein, was tested. Measurements were performed at T=22° C., using a cone on plate configuration (spindle C35/1 Ti Thermo HAAKE Rheo Stress 600 instrument). The obtained data is presented in FIGS. 10-13.

FIG. 10 presents comparative plots showing the viscosity of 5 mg/ml rhCollagen-MA (colMA) and 5 mg/ml rhCollagen-MA+Polyvinyl alcohol methacrylate (PVAMA) at rhCollagen-MA:PVAMA weight ratio of 5:1, 2:1, and 1:2.

FIG. 11 presents comparative plots showing the viscosity of 5 mg/ml rhCollagen-MA (colMA) and 5 mg/ml rhCollagen-MA+HAMA at rhCollagen-MA:HAMA weight ratio of 5:1 and 2:1.

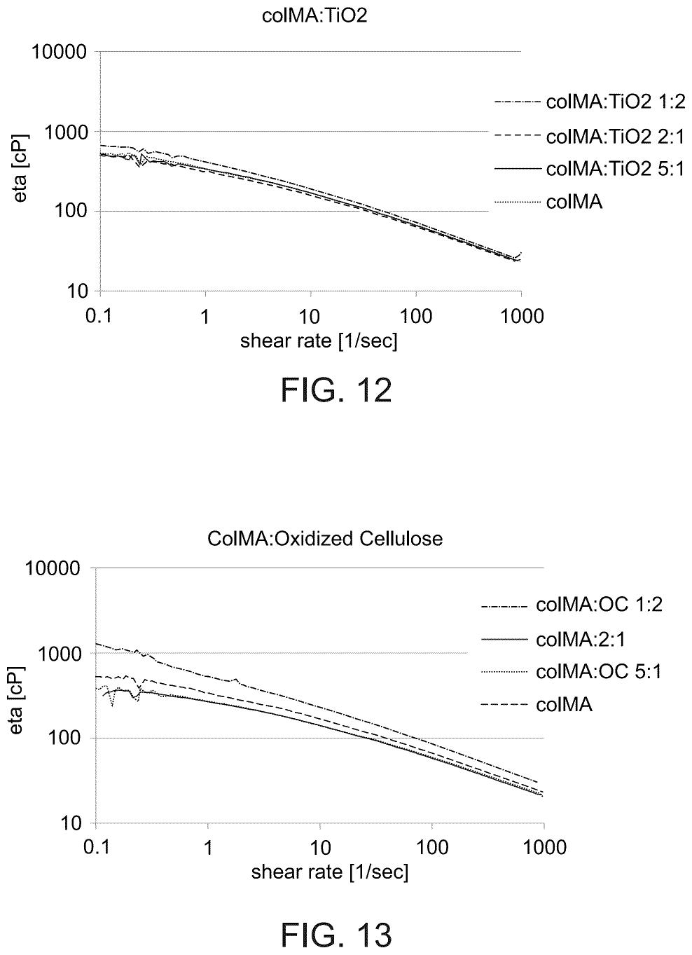

FIG. 12 presents comparative plots showing the viscosity of 5 mg/ml rhCollagen-MA (colMA) and 5 mg/ml rhCollagen-MA+TiO₂ at rhCollagen-MA:HAMA weight ratio of 5:1, 2:1, and 1:2.

FIG. 13 presents comparative plots showing the viscosity of 5 mg/ml rhCollagen-MA (colMA) and 5 mg/ml rhCollagen-MA+oxidized cellulose at rhCollagen-MA:HAMA weight ratio of 5:1, 2:1, and 1:2.

Cured rhCollagen-MA Formulations in 0.1 M Phosphate Buffer 0.1 M (pH 7.4)/NaCl 11.3 mM:

The above-described formulations, containing rhCollagen-MA alone or with the different additives at 2:1 collagen:additive weight ratio were mixed with the photoinitiator 2-Hydroxy-4′-(2-hydroxyethoxy)-2-methylpropiophenone (0.1%). 130 μL of the formulations were introduced in a silicon mold (8 mm×8 mm×8 mm) and illuminated for 20 seconds with UV light (365 nm) at a distance of 1.5 cm.

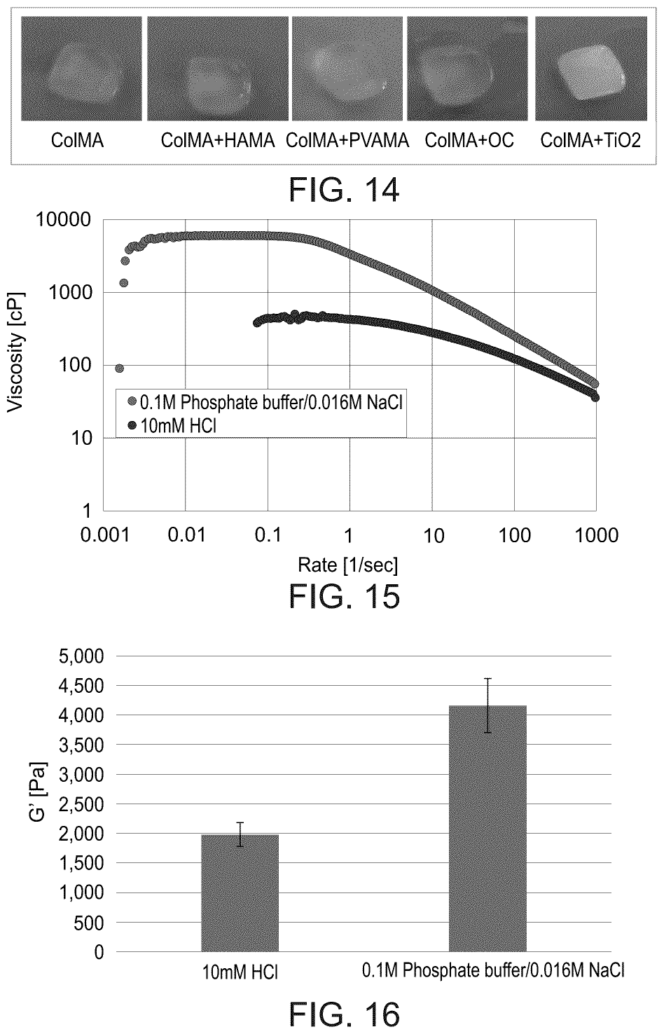

FIG. 14 presents images of the obtained mold objects.

Viscosity of rhCollagen-MA Formulations in Phosphate Buffer and in HCl and G′ Measurements of Cured Objects Made Therefrom:

10 mg/ml monomeric rhCollagen-MA (10-fold) solutions in phosphate buffer 0.1 M/NaCl 15.8 mM (about 16 mM) or in 10 mM HCl were prepared by adding the appropriate volume of the medium to the lyophilized material. After complete dissolution the formulation's viscosity was tested before curing.

The viscosity of these formulations at increased frequency was recorded_in a rheometer using Rotational CR mode at room temperature, and the obtained data is presented in FIG. 15. As shown therein, a 0.1 M phosphate buffer/0.016 M NaCl (total of 300 mOs/L) formulation presents higher viscosity (light grey) than a 10 mM HCl (dark grey) and 20 mM Acetic Acid (data not shown). The shear thinning behavior in this phosphate buffer medium is more pronounced compared with all other tested media.

These formulations were also subjected to curing at 365 nm by UV LED source. 1.6 ml of solution with 0.1% photoinitiator was injected into a discoid mold and curing was performed using 100 mW/cm² for 90 seconds, and G′ values at increased frequency of the cured objects was measured in a rheometer using Oscillation CR mode at 37° C.

The obtained data is shown in FIG. 16, indicating a higher storage modulus of the cured objects formed of a phosphate buffer-based formulation.

### Example 5

A liquid solution of 20 mg/ml, monomeric rhCollagen-MA (prepared using 10-folds molar excess of MA) in Hepes Buffered saline was mixed with N-vinyl pyrrolidinone and Eosin Y/TEA (triethanolamine) as a photoinitiator. 120 microliters were then dispensed in a silicon mold (8 mm×8 mm×3 mm) and covered with a mouse skin patch (Science in Action Ltd.). The formulation was polymerized transdermally by illuminating the external side of the skin patch with white light from a white LED torch for 6 minutes.

FIG. 17 presented a photograph, taken using a camera, of the skin patch following illumination, and show that the rhCollagen-MA polymerized and was integrated into the skin tissue.

### Example 6

Monomeric rhCollagen-MA alone and rhCollagen-MA (monomeric) combined with calcium phosphate particles were evaluated in a cell proliferation assay using Normal human dermal fibroblasts (Promocell).

Objects (scaffolds) were prepared from lyophilized rhCollagen-MA dissolved in DDW containing (1) rhCollagen-MA alone, 9 mg/ml; (2) rhCollagen-MA, 9 mg/ml and nano-hydroxyapatite (nanoHA), 7 mg/ml; and (3) rhCollagen-MA, 9 mg/ml and nano-tricalcium phosphate (nanoTCP), 7 mg/ml. Each solution was subjected to UV curing, lyophilized and sterilized with ethylene oxide (ETO). Normal human dermal fibroblasts were seeded on the scaffolds and cell proliferation was measured using colorimetric assay (WST-1) on day 1, 3 and 7. FIG. 18 presents the obtained data and demonstrate good cell proliferation in all tested formulations.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A formulation comprising a plant-derived recombinant human collagen featuring at least one curable group and an aqueous carrier, the formulation featuring a viscosity of no more than 2000 centipoises, or no more than 1,500 centipoises, at a shear strain rate of 5 1/sec, at room temperature, wherein a concentration of said plant-derived recombinant human collagen featuring said at least one curable group is at least 3 mg/mL.

2. The formulation of claim 1, wherein said at least one curable group comprises at least one photocurable group.

3. The formulation of claim 2, wherein said at least one curable group comprises at least one (meth)acrylic group.

4. The formulation of claim 3, wherein said plant-derived recombinant human collagen features a plurality of acrylamide or methacrylamide curable groups, each substituting an amine group of a lysine residue in said plant-derived recombinant human collagen.

5. The formulation of claim 4, wherein at least 50% of the lysine residues in said plant-derived recombinant human collagen are substituted by said methacrylamide or acrylamide group.

6. The formulation of claim 4, wherein at least 80% of the lysine residues in said plant-derived recombinant human collagen are substituted by said methacrylamide or acrylamide group.

7. The formulation of claim 1, wherein said plant-derived recombinant human collagen is a plant-derived recombinant human Type I collagen.

8. The formulation of claim 1, where said plant-derived recombinant human collagen is in a lyophilized form.

9. The formulation of claim 1, wherein said carrier comprises an acid.

10. The formulation of claim 1, wherein a concentration of said plant-derived recombinant human collagen featuring said at least one curable group ranges from 3 mg/mL to 50 mg/mL.

11. The formulation of claim 1, wherein a concentration of said plant-derived recombinant human collagen featuring said at least one curable group ranges from 3 mg/mL to 20 mg/mL.

12. The formulation of claim 1, further comprising at least one curable material other than said plant-derived recombinant human collagen featuring said curable group.

13. The formulation of claim 12, wherein a weight ratio of said recombinant human collagen featuring said curable group and said at least one curable material ranges from 10:1 to 1:2.

14. The formulation of claim 1, further comprising a biological material other than said plant-derived human recombinant collagen.

15. The formulation of claim 1, featuring a viscosity of no more than 2,000 centipoises, or no more than 1,500 centipoises, at a zero shear strain rate, at 37° C., wherein a concentration of said recombinant human collagen featuring a curable group is at least 3 mg/mL.

16. The formulation of claim 1, featuring, when hardened, storage modulus (G′) of at least 1,000 Pa.

17. The formulation of claim 1, featuring, when hardened, an increase of at least 10-folds of its storage modulus (G′).

18. The formulation of claim 1, usable in additive manufacturing of a three-dimensional object that comprises, in at least a portion thereof, a collagen-based material.

19. The formulation of claim 18, wherein said additive manufacturing comprises forming a plurality of layers in a configured pattern corresponding to the shape of the object, wherein at least a portion of said layers, are formed from the formulation at a temperature of at least 20° C.

20. The formulation of claim 18, further comprising at least one curable material other than said recombinant human collagen featuring said curable group; and/or an agent that modifies a mechanical and/or rheological and/or physical property of the formulation and/or of a respective portion of the object; and/or a biological material other than said human recombinant collagen.

21. The formulation of claim 1, wherein the formulation is hardened by exposing said portion of said layers to irradiation.

22. A kit comprising the formulation of claim 1 packaged therein.

23. The kit of claim 22, wherein said plant-derived recombinant human collagen featuring said at least one curable group and said aqueous carrier are packaged individually in the kit.

24. A kit comprising the formulation of claim 20 packaged therein.
